(19) United States
(12) Patent Application Publication
(10) Pub. No.: US 2012/0100604 A1
(43) Pub. Date: Apr. 26, 2012

(54) METHODS AND COMPOSITIONS FOR GENERATING SELF ATTENUATING GENETIC CIRCUITRY

(75) Inventors: Irinia M. Conboy, El Sobrante, CA (US); Morgan E. Carlson, Albany, CA (US); Michael Conboy, El Sobrante, CA (US)

(73) Assignee: THE REGENTS OF THE UNIVERSITY OF CALIFORNIA, Oakland, CA (US)

(21) Appl. No.: 13/130,984

(22) PCT Filed: Nov. 20, 2009
(86) PCT No.: PCT/US09/65376

§ 371 Date: Jan. 9, 2012

Related U.S. Application Data
(60) Provisional application No. 61/117,407, filed on Nov. 24, 2008.
(60) Provisional application No. 61/227,203, filed on Jul. 21, 2009.

(51) Int. Cl. C12N 5/10

(52) U.S. Cl. 435/325

(57) ABSTRACT

The invention provides compositions and methods for engineering self-attenuating circuitry in a cell that provides an optimal homeostasis between TGF-β/pSmad and Notch signaling to produce the long-term restoration of tissue regenerative potential and repair.

### CROSS-REFERENCE TO RELATED APPLICATIONS

This application claims priority to U.S. Provisional Patent Application No. 61/117,407, filed Nov. 24, 2008, and 61/227,203, filed Jul. 21, 2009, and the entire disclosure of both applications is herein incorporated in the entirety for all purposes.

### STATEMENT REGARDING FEDERALLY SPONSORED RESEARCH

This work was supported in part by a grant from the National Institutes of Health, grant number AG072252. The government may have some rights in this invention.

### FIELD OF THE INVENTION

The invention relates generally to the field of molecular biology, biochemistry, and cell biology of tissue repair and regeneration. The invention encompasses compositions and methods for producing a homeostasis of signaling in various pathways to affect long-term restoration of aged tissue repair. The present invention further encompasses compositions and methods for producing a homeostasis between TGF-β/pSmad and Notch signaling.

### BACKGROUND INFORMATION

The following is provided as background information only and should not be taken as an admission that any subject matter discussed or that any reference mentioned is prior art to the instant invention. All publications and patent applications herein are incorporated by reference to the same extent as if each individual publication or patent application was specifically and individually indicated to be incorporated by reference.

Organ regenerative capacity deterioration is largely attributed to age-related inhibitory changes within extrinsic cues that govern stem cell regenerative responses. Studies suggest that muscle stem cell regenerative responses are not triggered in the old due to a decline in Notch activation, and that these regenerative responses can be rejuvenated by forced local activation of Notch, by exposure to young circulation, and/or by factors produced in human embryonic stem cells. These studies suggest that stem cells endogenous to old organs have a capacity for maintenance and repair but are inhibited by an aged differentiated niche.

Studies also suggest that physiological aging of the differentiated muscle niche is defined by excessive TGF-β production, which interferes with old muscle stem cell regenerative responses by inducing heightened TGF-β/pSmad signaling and CDK inhibitor levels. Muscle repair success can be determined by an antagonistic balance between TGF-β/pSmad signaling levels and Notch pathway activation. (see Carlson et al., Nature, (2008), 454:528-532, which is herein incorporated in its entirety for all purposes, particularly for all teachings regarding muscle stem cell regenerative responses). However, in contrast to inhibitory aged levels, “young” levels of bio-active TGF-β do not interfere with regeneration-specific molecular signaling and muscle repair.

Methods are needed that can maintain and influence TGF-β/pSmad and Notch signaling homeostasis and thereby affect long-term restoration and repair of aged tissues. This invention described below addresses this need, as well as others.

### SUMMARY OF THE INVENTION

Accordingly, the present invention provides methods and compositions for engineering a self-attenuating circuitry for maintaining TGF-β/pSmad and Notch signaling homeostasis through variable recalibration of TGF-β/pSmad and Notch signaling intensities to improve stem cell regenerative potential and tissue repair.

In further embodiments, TGF-β/pSmad and Notch signaling homeostasis may be established and/or maintained by direct recalibration of TGF-β/pSmad and Notch signaling and/or through indirect effects on one or both signaling pathways through manipulation of other pathways, such as MAPK.

Other objects, aspects and advantages of the instant invention are set forth in the detailed description which follows.

### BRIEF DESCRIPTION OF THE DRAWINGS

FIG. 1 is a schematic illustration of an exemplary gene regulatory circuitry of the invention showing interactions with endogenous TGF-β and Notch signaling pathways.

FIG. 2 provides experimental results showing that advancing age TGF-levels increase in circulation of mice (A); and also increase locally in muscle tissue of elderly individuals (B).

FIG. 3A is a schematic illustration of an embodiment of the present invention; 3B provides experimental results from skeletal muscle cryosections.

FIG. 4 provides immunofluorescence data for old and young skeletal muscle cryosections in which (A) shows immunodetection of embryonic myosin heavy chain (eMyHC) and dystrophin with Hoechst labeled nuclei; (B) shows negative staining control with isotype-matched primary antibodies; (C) shows additional H & E images; (D) shows quantification of scar tissue formation; (E) shows a demarcated H&E image, and (F) shows quantification of area of regeneration versus degeneration.

FIG. 5 provides experimental results showing myogenic cell expansion in human muscle that undergoes exercise after immobility. (A) shows cryosections immunostained for Pax7, M-Cadherin, and NCAM. (B) shows total numbers of satellite cells quantified per 10 mm² for both young and old muscle. (C) shows Pax7+ nuclei per 10 mm² for both young and old muscle. (D) shows Western Blot results for Pax7 on whole muscle protein isolates, which are quantified in (E).

FIG. 6 provides experimental results showing that Notch activation and Delta upregulation is diminished in regenerating old human skeletal muscle. (A) shows immunostained cryosections stained for co-expression of nuclear Pax7+/active Notch in resident satellite cells. (B) shows Delta and dystrophin immunodetection in 10 μm skeletal muscle cryosections. (C) shows Western Blot of Notch and Delta levels in whole muscle protein isolates, which are quantified in (D). (E) is a bar graph quantifying Notch/Pax7 double-positive myofiber-associated cells from cryosections.

FIG. 7 provides experimental results from immunodetection of: (A) TGF-β and laminin; (B) P-Smad3 and laminin; (C) Western blots for TGF-β, P-smad3, p15, p21, p16 and p27 from whole muscle protein isolates, which are quantified in (D). (E) shows activated satellite cells cultured for 24 hours in OPTIM-MEM containing age-matched human sera in the presence of recombinant TGF-β1. (F) is a bar graph quantifying myogenic responses based on co-expression of desmin/BrdU.

FIG. 8 provides experimental results showing (A) activated satellite cells cultured for 24 hours in the presence of recombinant TGF-β1 and then transferred to Differentiation Medium (DMEM+2% Horse Serum) for 72 hours; (B) a bar graph showing myogenic differentiation index based on expression of eMyHC; and (C) Immunostaining of myogenic cell populations derived from isolated satellite cells.

FIG. 9 provides experimental results showing (A) Western blot on whole muscle protein isolates for P-Erk and Erk, which are quantified in (B). (C) shows Western blot of Notch, Delta, P-Erk and Erk, which is quantified in (D). (E) shows activated satellite cells analyzed for myogenic responses, which are quantified in (F) based on co-expression of desmin/BrdU.

FIG. 10A is a bar graph quantifying myogenic expansion and differentiation of isolated satellite cells from young and old human subjects cultured in the presence of young versus aged blood sera (isochronic and heterochronic). (B) shows quantification of myogenic activation and expansion based on immunostaining for Pax7 and desmin.

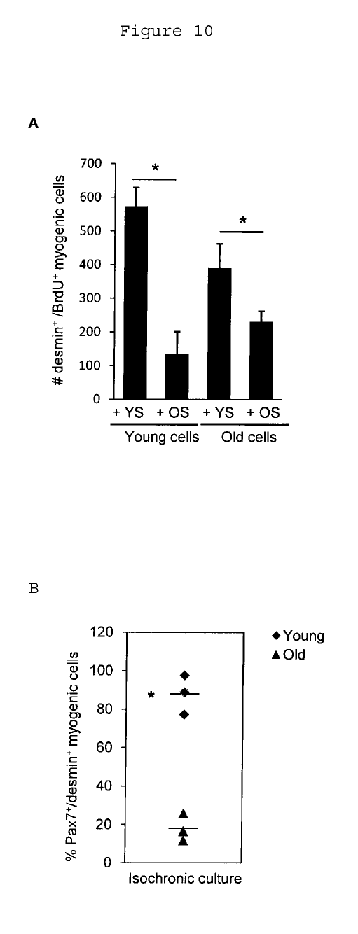

FIG. 11 shows (A) activated satellite cells cultured for 24 hours in the presence of GSI or GSI+recombinant human FGF and (B) quantification of myogenic responses based on co-expression of desmin/BrdU.

FIG. 12 shows (A) images from young and old satellite cells isolated and cultured in the presence of young or old sera with or without experimentally-induced Notch activation; (B) quantification of the images in (A); (C) Western blot analysis for expression of p15 and p21; and (D) quantification of the results in (C).

FIG. 13A is a bar graph showing results from ELISA analysis of TGF-β1 levels in the serum of aged humans (AG: 65-90 years old) versus young (YG: 20-35 year old). Error bars (s.e.m.) represent the mean of 53 different subjects in each group; p<0.0001. (B) is a bar graph showing that mouse serum TGF-β1 becomes elevated with age. (C) and (D) show age-dependent curves of TGF-β levels. Dot plots represent separate individuals (C) and separate animals (D) of indicated ages. (E) and (F) show levels of Wnt in human and mouse sera respectively. Inset panel shows 0.1-10 ng/ml Wnt3a range in greater detail.

FIG. 14 shows: (A) data from myofiber explants cultures, grown for 24 and 48 hours in isochronic (Y—Y, O—O) and heterochronic (Y—O, O—Y) conditions. Data depicts representative profile of desmin+/BrdU+cells (% myogenic proliferation), shown as mean %+/−S.D; (B) data from myofiber explants, cultured for 48 and 72 hours under differentiation-inducing conditions. Data depicts representative profile of eMyHC-associated nuclei (% myogenic differentiation), shown as mean %+/−S.D. (C) and (D) show profiles of data (shown in A and B) of expected reduction in myogenic proliferation from 24 hours to 48 hours versus increases in degree of differentiation from 48 to 72 hours.

FIGS. 15(A) and (B) are bar graphs showing % myogenic proliferation in young and old myofiber-associated myogenic progenitor cells isolated 3 days post injury and cultured overnight in Opti-MEM containing either 10% young serum (YS), 10% old (OS), TGF-β1 antibody depleted serum alone, or with fixed amounts of recombinant TGF-β1 Cells were cultured with their specific sera for 24 hrs (A and B), and transferred to differentiation medium for additional 48 hours (D and E). (C) shows images from cells fixed and fixed and immunostained for desmin and BrdU, with Hoechst marking all nuclei.

FIG. 16 shows images from myofiber-associated myogenic progenitor cells isolated 3 days post injury and cultured overnight in Opti-MEM containing (A.) 10% young serum (YS); YS+Wnt3A; YS+Wnt3A+TGF-β; or (B) 10% old (OS), OS+FRP3, OS+FRP3+anti-TGF-β. BrdU was added for the last 2 hours to measure proliferation. (C) shows quantification of results in (A) and (D) shows quantification of results in (B). (E) is a bar graph of cells scored from the assays of (A)-(D), with results displayed as the mean percent of desmin+ve myotube associated nuclei/total nuclei, +/−S.D. (F) is a Western blot of young and old satellite cells isolated from 3 day post-injury myofibers explants and cultured overnight in Opti-MEM containing 10% isochronic sera analyzed for levels of phospho-tyrosine GSK3β, phosphor-serine GSK3β and total GSK3β levels. Actin was used a loading control. (G) is a bar graph quantifying the results from (F).

FIG. 17 shows results from myofibers with activated satellite cells obtained from young and old mice injured with cardiotoxin. (A) shows images from myofiber-derived cells cultured for 48 hours in OPTI-MEM+5% young (YS) or old (OS) mouse serum. (B) shows quantification of regenerative potential shown in A. (C) shows levels of nuclear P-Smad3 in old satellite cells transduced with DN RII-expressing lentivirus, in vitro. Old satellite cells, exposed to old serum (OS) plus control virus, or old serum plus DN RII-expressing virus (as indicated), were immunostained for P-Smad3.

FIG. 18 shows results from experiments on old or young mice injected (subcutaneous) with a small molecule inhibitor of the TGF-β RI Kinase (Ki), for 2 weeks. Five days before the end of treatment, muscle was injured. At the end of treatment, animals were sacrificed and muscle was collected. BrdU was injected (intraperitoneal) at 3 days post-injury to label proliferating, fusion-competent myoblasts. (A) shows images of cryosections (10 μm) from young and old muscle (receiving vehicle alone), Old+Ki and Old+RII. (B) shows quantification of regeneration presented as mean percent of newly-regenerated myofibers per square millimeter of injury site.

FIG. 19 shows (A) images from myofibers isolated from young control and kinase inhibitor treated animals; (B) quantification of regeneration from muscle sections, and is presented as mean of newly-regenerated myofibers per square millimeter of injury site; (C) analysis of TGF-β serum levels in Young+Ki animals as compared to young; and (D) quantification of regeneration from muscle sections, presented as mean of newly-regenerated myofibers per square millimeter of injury site.

FIG. 20 shows: (A) Images of myofibers isolated from young and old mice treated for TGF-β systemic down-modulation; (B) quantification of results of multiple Western blot assays (normalization of TGF-β and P-Smad3 pixel density by actin-specific pixel density) depicted by relative pixel intensity; (C) quantification of TGF-β levels in serum for each animal in young, old or old+experimental treatments as determined by ELISA.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### Definitions and Abbreviations

The abbreviations used herein generally have their conventional meaning within the chemical and biological arts.

The term “autologous cells”, as used herein, refers to cells which are an organism's own genetically identical cells.

The term “heterologous cells”, as used herein, refers to cells which are not an organism's own and are genetically different cells.

The term “stem cells”, as used herein, refers to cells capable of differentiation into other cell types, including those having a particular, specialized function (i.e., terminally differentiated cells, such as erythrocytes, macrophages, etc.). Stem cells can be defined according to their source (adult/somatic stem cells, embryonic stem cells), or according to their potency (totipotent, pluripotent, multipotent and unipotent).

The term “unipotent”, as used herein, refers to cells can produce only one cell type, but have the property of self-renewal which distinguishes them from non-stem cells.

The term, “multipotent”, or “progenitor”, as used herein, refers to cells which can give rise to any one of several different terminally differentiated cell types. These different cell types are usually closely related (e.g. blood cells such as red blood cells, white blood cells and platelets). For example, mesenchymal stem cells (also known as marrow stromal cells) are multipotent cells, and are capable of forming osteoblasts, chondrocytes, myocytes, adipocytes, neuronal cells, and β-pancreatic islets cells.

The term “pluripotent”, as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent”/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells.

The term “totipotent”, as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive. An example of dedifferentiation is the conversion of a myogenic progenitor cell, such as early primary myoblast, to a muscle stem cell or satellite cell.

The term “anti-aging environment”, as used herein, is an environment which will cause a cell to dedifferentiate, or to maintain its current state of differentiation. For example, in an anti-aging environment, a myogenic progenitor cell would either maintain its current state of differentiation, or it would dedifferentiate into a satellite cell.

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “growth environment” is an environment in which stem cells will proliferate in vitro. Features of the environment include the medium in which the cells are cultured, and a supporting structure (such as a substrate on a solid surface) if present.

The term “regenerative capacity”, as used herein, refers to conversion of stem cell into dividing progenitor cell and differentiated tissue-specific cell.

The term “rejuvenation”, as used herein, refers to changing the regenerative responses of a stem cell such that the stem cell successfully or productively regenerates tissues in organs even if such organs and tissues are old and the stem cells are old.

The term “MAPK” refers to “mitogen-activated protein kinase” and as used herein can refer to the protein or its signaling pathway.

### Introduction

The present invention provides methods and compositions for establishing an engineered, tunable gene regulatory system for stabilizing the youthful profile of key signal transduction pathways that are responsible for efficient tissue maintenance and repair. This system is represented by self attenuating genetic circuitry that is able to (1) detect signaling deregulation and (2) restore the optimal homeostatic signaling intensity by down-modulation of TGF-β/pSmad signaling and upregulation of Notch activity in aged and inflamed cells and tissues, thereby boosting tissue regeneration. This self-attenuating circuitry can be engineered in vectors, such as viral vectors, that are able to introduce synthetic regulation into cells (either in vitro or in vivo).

In one embodiment, the present invention provides methods and compositions for recalibrating TGF-β/pSmad and Notch signaling through a tunable delivery of antagonists of TGF-β/pSmad and agonists of Notch signaling to a cell. In further embodiments, tunable delivery of antagonists of TGF-β/pSmad and agonists of Notch signaling to a cell involves a tuning or recalibration of signaling intensities.

In further embodiments, targeting the MAPK/pERK pathway either by itself or in combination with the manipulations of the TGF-β/pSmad and Notch homeostasis described herein can also be used to promote and/or stabilize tissue maintenance and repair at youthful levels.

Vectors of use in the present invention are designed for precise recalibration of TGF-β/pSmad and Notch signaling intensities in cells, including stem cells. These vectors establish this recalibration by expressing (1) a dominant negative form of TGF-β receptor type II and/or (2) the Notch ligand Delta. This expression is generally under the control of a promoter, such as a tetracycline-inducible pSmad3 responsive promoter element.

In the engineered genetic circuitry established by the present invention, the more active pSmad3 is in the nucleus, the higher is the expression of the TGF-β/pSmad inhibitory receptor and/or Notch activation by ectopic Delta expression. This network thus responds to the excessive TGF-β/pSmad levels typical of aged/inflamed tissues by attenuating the TGF-β/pSmad signaling strength and/or by activating Notch. At the same time, induction under a promoter, such as a tetracycline promoter, allows deliberate control over TGF-β/pSmad and/or Notch signaling intensities. Forced activation of Notch or attenuation of TGF-β/pSmad restore regenerative responses to stem cells residing in old muscle through down regulation of cyclin-dependent kinase (CDK) inhibitors, such as p15, p16, p21 and p27, and thus the self-attenuating circuitry established by the present invention produces long-term restoration of aged/pathologic tissue repair.

A novel feature of the present system is the ability to precisely recalibrate the optimal “youthful” strength of signal transduction pathways through the built-in detection of the physiological age and/or inflammation state of a given cell. Such detection is based on Smad-responsive promoter elements (SRE) of the synthetic gene regulatory circuitry. This is in contrast to conventional methods, which are based on turning genes and pathways “on” or “off” with little to no control of signaling strength in these pathways. The present invention provides methods and compositions that can effectively tune signal transduction pathways, including TGF-β/pSmad and Notch signaling pathways, to (1) detect deviations of endogenous pathways from their youthful signaling strength and (2) restore and maintain youthful levels of signaling.

The present invention also provides self-attenuation feature by which down-modulation of TGF-signaling by the actuator, e.g. dominant negative TGF-β receptor resulting in lower pSmad3 levels will be detected by SRE and in turn, tune down the expression of the dominant negative TGF-β receptor, therefore maintaining TGF-β/pSmad signaling at homeostatic levels.

The present invention also provides a safety switch feature, since pSmad3-induced SRE promoter is driving the expression of tetracycline activator protein, which in turn induces the expression of actuators, e.g. TGF-β dominant negative receptor and/or Notch ligand Delta under the control of tetracycline responsive promoter (TRE) only in the presence of a small molecule, such as doxicyclin.

Additional optional features of this invention are IRES-GFP detection block by which expression of green fluorescent protein is simultaneous with the expression of actuators, such as TGF-β dominant negative receptor and/or Notch ligand Delta under the control of tetracycline responsive promoter (TRE); this feature allows assess the successful function of the synthetic circuitry.

The present invention also provides novel designer viral cassettes that utilize interchangeable and unique combinations of genes and promoters within a single system, thereby enabling focused expression of a synthetic gene regulatory system for tunable recalibration of TGF-β/pSmad and Notch pathways, in order to maintain effective signaling homeostasis within any specific cell, including stem cell subsets of interest. In this manner, the self-attenuating circuitry established using methods and compositions of the present invention provide a way to maintain productive regenerative responses in muscle, liver, brain, etc., regardless of the age of the tissue and the pathology of the tissue in which the regenerating cells reside.

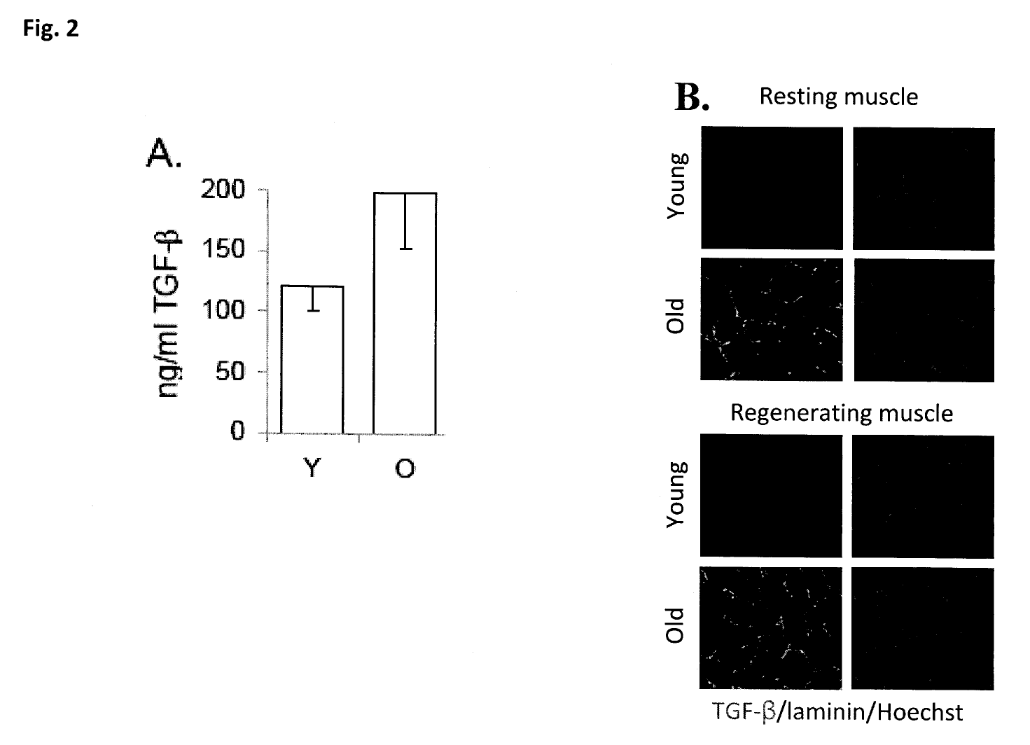

Although for clarity much of the following discussion is provided in terms of muscle cells, it will be appreciated by one of skill in the art that the methods and compositions described herein are applicable to a wide range of cells, including stem cells other than muscle stem cells. Additionally, TGF-β and Notch are known to function in most cells, and the inventors have found that aging mechanisms are conserved between mouse and human (see FIG. 2).

In further embodiments, the present invention also provides methods and compositions for manipulating the MAPK pathway and thereby affecting Notch signaling. As is described herein, the MAPK pathway is an age-responsive positive regulator of Notch in human muscle, and manipulation of the MAPK pathway can be used alone or in combination with any of the methods described herein to affect signaling pathways associated with young tissue.

In still further embodiments, the present invention provides methods and compositions for enhanced and rejuvenated tissue repair by systemic delivery of TGF-β antagonists, including small molecules that reach all organs via blood circulation).

### Tunable Constructs

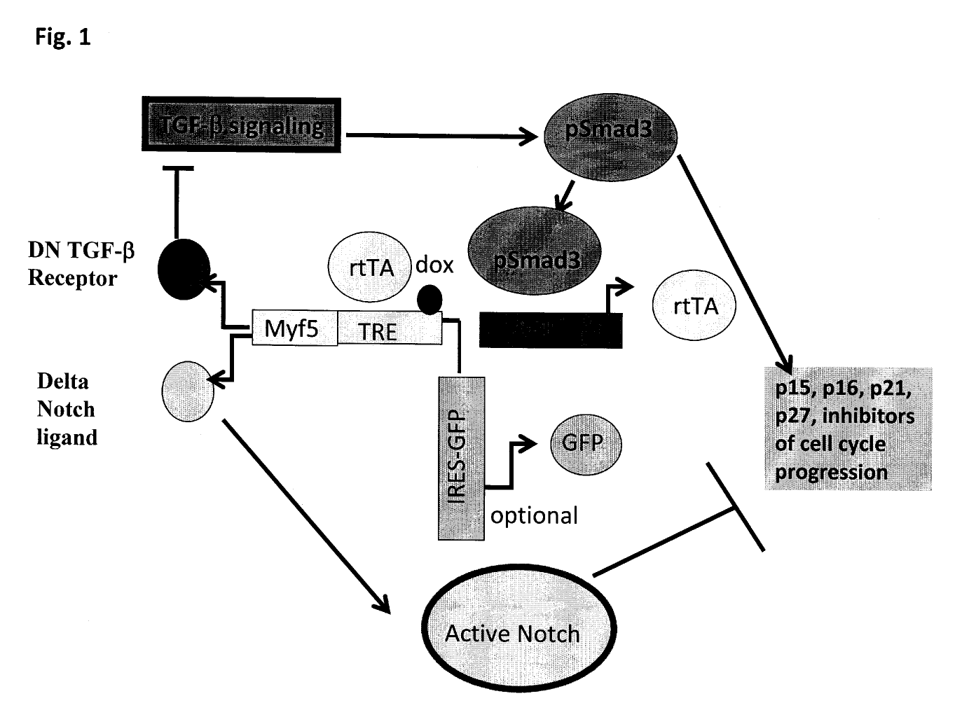

In one aspect, the invention provides tunable constructs for inducible expression of TGF-β dominant negative receptors and/or Notch ligand Delta. In an exemplary aspect, the invention provides a viral construct for tetracycline-inducible expression of TGF-β dominant negative receptor and/or Notch ligand. In a further exemplary embodiment, the construct contains TGF-β DNRII or Notch ligand, Delta, under tet-inducible promoter (TRE) control and the tet-responsive transactivator protein cloned into a viral backbone (see FIG. 1 for a schematic illustration of elements of an exemplary embodiment of tunable constructs of the invention).

In an exemplary embodiment, the construct is formed by inserting TGF-β DN RII, or Delta, into the entry vector pENTR (Invitrogen), allowing one-step recombination cloning into the destination vector, pLenti/TO/V5-DEST (Invitrogen) under TRE control. The tet-responsive trans-activator protein (rtTA) will be sub-cloned into pIRES-eGFP vector (Clontech), thus producing a CMV-rtTA-pIRES-eGFP fragment, which will in turn be inserted into pLenti/TO/V5-DEST to produce the final construct: CMV-rtTA-pIRES-eGFP-TRE-TGF-β RII DN/Delta. GFP will be expressed as a distinct transcript, with rtTA (IRES controlled), and used to monitor expression efficiency.

It will be appreciated that not all of the above described elements of a tunable constructs must be present in order to produce the effects described herein. For example, the GFP element is optional and in further embodiments may comprise elements encoding for other detectable signals.

In some embodiments, defined promoter regions (PROM in FIG. 1) for response to excessive TGF-β signaling (pSmad3 response element) can be used to replace CMV (ubiquitous expression). Expression of TGF-β DN RII inhibitory receptor, or Notch activation via ectopic Delta, will thus become a function of nuclear pSmad3 levels. TGF-β DN RII inhibitory receptor will attenuate TGF-β signaling strength, while activated Notch will antagonize the induction of physiological inhibitors of cell cycle progression, e.g. p5, p16, p21 and p27 by TGF-β/pSmad3.

In other embodiments, defined promoter region tetracycline responsive element (TRE) in FIG. 1 induces the expression of TGF-β DN RII inhibitory receptor, or Delta when (1) tetracycline homolog doxicycline (dox) is added and (2) excessive TGF-signaling, e.g. elevated pSmad3 induces the expression of tetracycline activator protein (rtTA); doxicycline thus, is a “safety switch” removal of which will turn off the circuitry.

In a further embodiment, promoter sites are used for stem cell-specific expression, including without limitation the Myf-5 promoter in muscle stem cells. Promoters in other stem cell types, e.g. nestin for neural lineage, and the like can be used if the circuitry provided by the present invention is used in other organs, including but not limited to muscle. Similarly, promoters bound by other age-specific factors, e.g. NF-kB could be used to introduce attenuation of their respective signaling pathways.

In a still further embodiment, final expression constructs are co-transfected into cells, including without limitation 293FT packaging cells. Viral supernatants can be harvested, titered and used for transducing non-dividing stem cells (such as non-injured muscle) and proliferating stem cells (such as those activated by injury). During the first days after injury, satellite cells need to break quiescence and proliferate to repair the injury. However, there is an age-specific elevation of pSmad3 and diminished Notch activation, either of which can inhibit cell-cycle progression. The present invention, by altering the signaling of pSmad3 and/or Notch, can provide the ability to affect signaling intensity levels in both non-injured and proliferating cells.

A network established by constructs such as those described above will respond to excessive TGF-β/pSmad levels of the aged niche by attenuating its own signaling strength and/or activating Notch—either action will restore regenerative responses to aged stem cells.

### Recalibration

Viral constructs such as those described above can be used to infect tissues using methods known in the art. Tissues infected with such constructs can be recalibrated to maintain a self-attenuating circuitry that will lead to an optimal TGF-β/pSmad and Notch homeostasis. This homeostasis will then promote long-term restoration of aged tissue repair.

In one aspect, viral supernatants such as those described above will be used to infect tissue. Vector delivery will be based on plasmid DNA, lentiviral/retroviral, or AAV, which has been shown to provide sustained transgene expression in numerous tissues, including muscle. In vivo infection can in some embodiments be optimized to yield the highest transduction rate with the pSmad response element and with stem cell-specific promoter constructs. In further embodiments, a CMV-driven rtTA construct will be informative as giving high expression levels of TRE-driven TGF-β DN RII/Delta, thus turning the synthetic circuitry maximally “on” since aged muscle contains many resident cell types that become exposed to excessive TGF-β levels. Therefore, attenuating levels of TGF-β/pSmad signaling in cells, including satellite cells and other muscle cells, will mimic broader physiological “rejuvenation” of the muscle niche.

In an exemplary embodiment, after infection with the above described vector has resulted in stable expression, varying doses of doxycycline can be administered by direct oral administration. Doxycycline is a potent tetracycline analog that can induce a range of TGF-β DN RII/Delta expression. In a further embodiment, about 1 to about 50 μg/ml of doxycycline will be used. The varying doses of doxycycline can in some embodiments be normalized per body weight. As will be appreciated, other methods of administration can include without limitation oral, parenteral, inhalation or spray or rectal administration in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like may be of particular use in methods of the present invention.

In animals (or cultured tissues) expressing tunable vectors of the invention, administering doxycycline will reduce the levels of TGF-β/pSmad signaling and/or induce Notch activation, thus providing a self-attenuating circuitry that is under the deliberate control switch of doxycycline manipulation for transgene activation. In cases where excessive hyper- or hypo-activation of Notch/TGF-β/pSmad signaling would be physiologically detrimental, these internal mechanisms (pSmad3 responsive/self attenuation and doxycycline responsive control) ensure youthful balance restoration. Precise in vivo doses of doxycycline will in some embodiments be dependent on the age and/or the inflammatory state of the tissue in need of repair. In further embodiments, the doses of doxycycline applied will be based at least in part on in vitro tests. Such in vitro tests can include with out limitation Western Blotting or ELISA to measure TGF-b/pSmad3 and Delta production by old cultured cells in their own old niches (sera or myofibers) in the presence of the regulatory gene circuitry under variable levels of Dox; Dox concentrations resulting in the “young” levels of TGF-b/pSmad3 and active Notch will be used in vivo. FIG. 2A demonstrates that by ELISA TGF-b circulating levels are elevated in old mice. As will be appreciated by on of skill in the art, the above methods and compositions for recalibrating Notch and TGF-β/pSmad signaling in vivo may also be applied to cultured cells and tissues in vitro. In a further embodiment, cells and tissues treated in vitro using methods and compositions described above to recalibrate signaling pathways may be transplanted into organisms to affect signaling pathways in vivo. FIG. 2B shows by immuno-fluorescent detection that there is excessive TGF-β (green) surrounding aged human muscle fibers in vivo, laminin (red) delineates muscle fibers and Hoechst (blue) labels all cell nuclei.

### Attenuating TGF-β Levels

In accordance with any of the methods described herein, the present invention further provides methods and compositions for attenuating TGF-β levels for rescue of myogenic responses in old serum in vitro and in old animals in vivo. An unexpected result of the experiments described herein is that Wnt is not an age-specific systemic inhibitor of satellite cell myogenicity and can antagonize the inhibitory influence of TGF-β1 on myogenesis. Although the following discussion is in terms of TGF-β1, it will be appreciated that the following methods and compositions can be altered using methods known in the art to manipulate the levels of any member of the TGF-β super-family.

In some embodiments, the present invention provides methods and compositions for rejuvenating aged muscle repair by attenuating the TGF-β pathway through systemic pharmacological intervention in vivo.

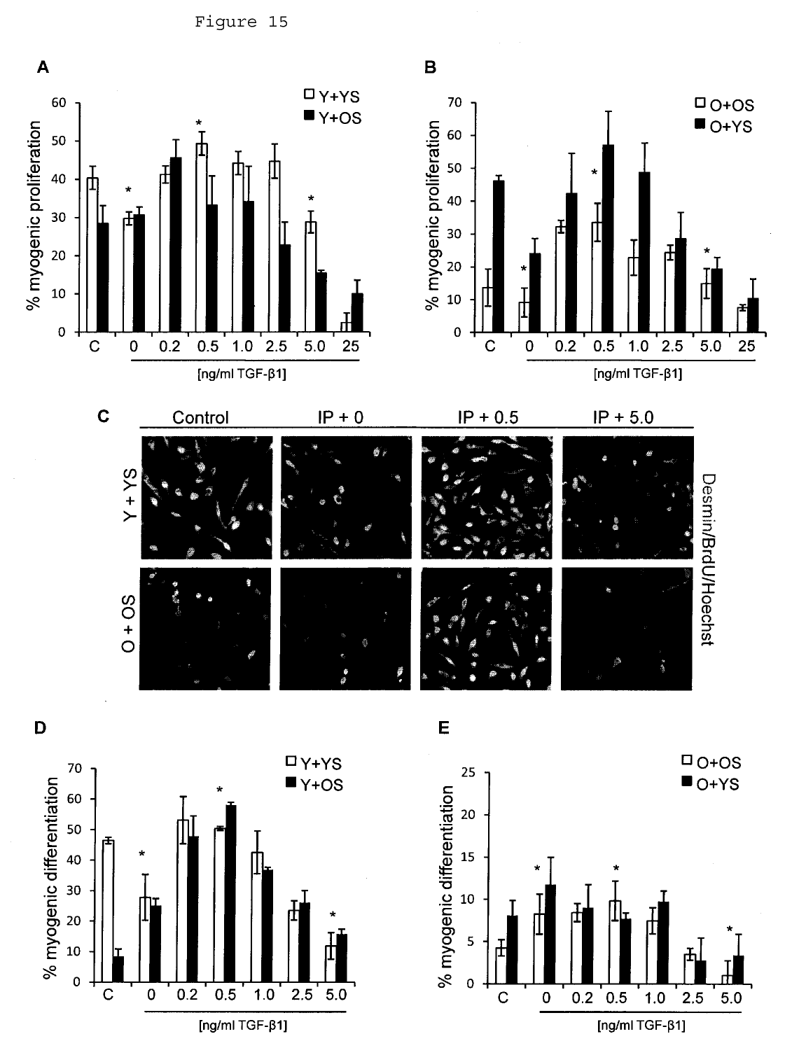

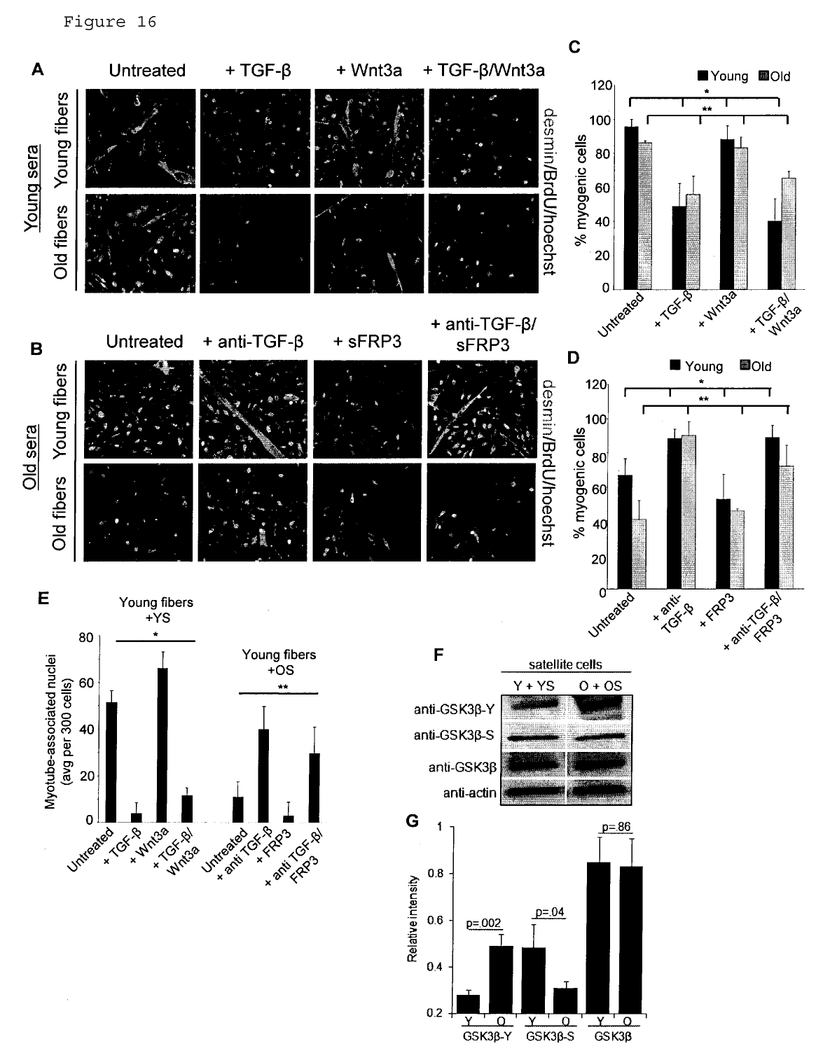

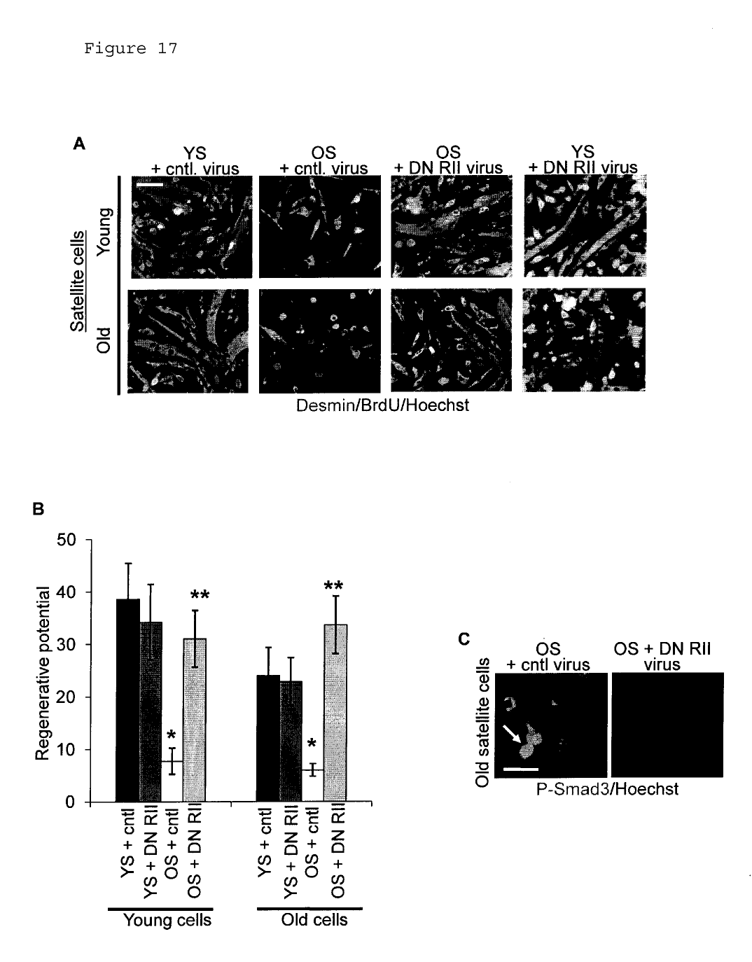

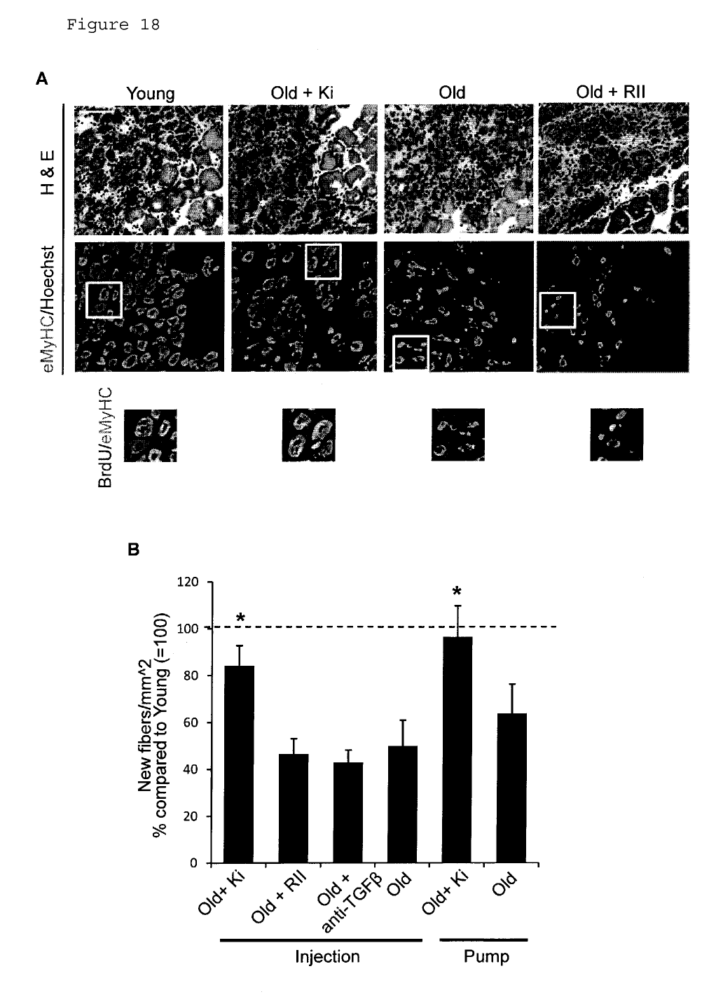

Elevated TGF-β1 in serum directly reduces the activation of satellite cells (FIG. 15), operating in a threshold fashion (FIGS. 17 and 16) and appears to function through the TGF-β II receptor and canonical pSmad signaling (FIGS. 18-20). Considering that TGF-β1 is broadly produced and signals to a variety of cells, the age-specific, evolutionarily conserved elevation of its functional levels in the old circulation might affect stem cell responses in many tissues and not just in skeletal muscle, and thus provides an explanation for the general decline of regenerative potential with advancing age in mammals, including without limitation mice and humans. As such, the methods and compositions of the present invention are applicable to tuning regeneration in many tissues and is not limited to skeletal muscle.

In some embodiments, the present invention provides methods in which the application of small molecules is used to modify TGF-β1 levels and calibrate the TGF-β pathway to young levels. In further embodiments, such small molecules may include without limitation a kinase inhibitor. In still further embodiments, the kinase inhibitor is a TGF-β RI kinase inhibitor.

In some embodiments, a TGF-β dominant-negative receptor II (TGF-β DN RII) is used to affect levels of TGF-β1, as described in further detail herein.

In further embodiments and in accordance with any of the above, method of the invention include application of intra-muscular RNAi to Smad3, to lower the levels of cytokine produced by muscle cells. Such an application may be conducted simultaneously with or in series with any of the methods and compositions described herein.

### Exemplary Embodiments

In one aspect, the present invention provides methods for enhancing the regenerative potential of a cell. Such methods include recalibrating TGF-β/pSmad and Notch signaling intensities in the cell.

In a further embodiment and in accordance with the above, recalibrating TGF-β/pSmad and Notch signaling intensities in the cell comprises introducing a tunable viral construct into the cell.

In a still further embodiment and in accordance with any of the above, tunable viral constructs of use in the present invention include TGF-β DN RII and/or Notch ligand, Delta; a tet-inducible promoter; and a tet-responsive trans-activator protein.

In further embodiments and in accordance with any of the above, the cells targeted with methods and compositions of the invention are stem cells. In a still further embodiment, the stem cells are muscle stem cells.

In a further embodiment and in accordance with any of the above, the promoter used in viral constructs of the invention is stem cell-specific. In a still further embodiment, the promoter is a Myf-5 promoter.

In a still further embodiment and in accordance with any of the above, TGF-β/pSmad and Notch signaling intensities in a cell includes increasing Notch signaling and attenuating TGF-β/pSmad signaling.

In a further embodiment and in accordance with any of the above, The method of claim 8, the increasing of Notch signaling and the attenuating of TGF-β/pSmad signaling are in homeostasis, such that the increasing of Notch signaling results in a concomitant attenuating of the TGF-β/pSmad signaling, and the attenuating of said TGF-β/pSmad signaling results in a concomitant increasing of the Notch signaling.

In a still further embodiment and in accordance with any of the above, doxycycline is used as a control switch, such that applying doxycycline to the cell is used to alter levels of Notch and TGF-β/pSmad signaling.

In a still further embodiment and in accordance with any of the above, manipulation of MAPK signaling using methods known in the art and described herein can be used in calibration of signaling homeostasis to produce and/or maintain regenerative potential of cells.

In a still further embodiment and in accordance with any of the above, transplanted tissue, particularly stem cell-based transplants, are modified using methods described herein to neutralize inhibitory effects of age-related processes and maintain regenerative potential of the transplanted cells.

In a yet further embodiment and in accordance with any of the above, levels of TGF-β1 are manipulated to affect regenerative potential of cells. In a still further embodiment, attenuating TGF-β1 in old sera is used to rejuvenate myogenic responses, while adding TGF-β1 to young sera “ages” myogenesis.

### EXAMPLES

### Example I

### Confirmation of Regulation of Signaling by Genetically Engineered Circuitry

Cells are infected with viral supernatants and varying doses of doxycycline to induce a range of TGF-β DN RII/Delta expression. 1-10 μg/ml of doxycycline are tested based on preliminary dose response studies.

Down-regulation of TGF-β/pSmad signaling intensity are confirmed by diminished levels of pSmad3 in cells exposed to aged serum, or when exogenous TGF-β1 is added. Cell cultures with control levels of TGF-β/pSmad signaling (no doxycycline), and with attenuated TGF-β (with 1-10 μg/ml of doxycycline) are compared in their myogenic potential (based on percent generation of proliferating, desmin+myoblasts that fuse into new MyHC myotube progenitor cell colonies). Desmin is a protein expressed at high levels in myoblasts, and MyHC is a protein expressed at high levels in newly formed myotubes. Generation of myoblasts and myotubes are key steps in muscle regeneration. Levels of active Notch and TGF-β/pSmad are determined in these cultures as a function of variable doxycycline via Western blot. In this system, the more active pSmad3 is in the nucleus, the higher will be the expression of rtTA which in the presence of doxycycline will attenuate TGF-β/pSmad signaling and/or induce activation of Notch via ectopic expression of Delta. Levels of active Notch are determined in these cultures as a function of variable TGF-β/pSmad and Notch signaling strength via Western blot. In this system, the more active pSmad3 is in the nucleus, the higher will be the expression of TGF-β/pSmad and/or Notch signaling intensities. Either forced activation of Notch or attenuation of TGF-β/pSmad restores regenerative responses to stem cells residing in old muscle. The self-attenuating circuitry aimed to maintain optimal TGF-β/pSmad and Notch homeostasis is tested for the long-term restoration of aged tissue repair.

### Example II

### Muscle Isolation and Satellite-Cell Culture

Myofiber explants and satellite cells were generated from C57 BL/6 mice. Whole muscle underwent enzymatic digestion at 37° C. in DMEM (Invitrogen)/1% penicillin-streptomycin/125 U ml⁻¹. Collagenase Type IIA solution. Bulk myofibers with associated satellite cells (located beneath the basement membrane and above the plasma membrane) were purified away from muscle interstitial cells, tendons, etc., by multiple rounds of trituration, sedimentation and washing. Satellite cells were isolated from purified myofibers by subsequent enzymatic digestion with Collagenase Type IIA and Dispase, followed by sedimentation, washing and fine-mesh straining procedures. The approximate 95% purity of satellite cells was routinely confirmed by generation of proliferating fusion-competent myoblasts after 24 hours in growth medium (Ham's F-10 (Mediatech), 20% FBS (Mediatech), 5 ng/ml FGF (Chemicon) and 1% penicillin-streptomycin); and myotube formation after 48 hours in DMEM+2% horse serum. The efficiency of satellite-cell depletions was confirmed by the absence of such myogenic potential. Satellite cells were cultured on ECM:PBS-coated plates.

### Example III

### shRNA Delivery by Lentiviral Transduction

Young and old tibialis anterior and gastrocnemius muscles were infected, in vivo, with control non-target shRNA (Sigma SHC002V), GFP (Sigma SHC003V) or pLKO.1-puro (Sigma SHC001V) control transduction lentiviral particles (≧10⁶ TU/ml, as determined by p24 antigen ELISA titer). Mouse Smad3 shRNA-producing lentiviral particles (also obtained from Sigma) were used for in vivo transduction experiments [Target-set generated from accession number NM_{—}016769.2:

(1) CCGGCCCATGTTTCTGCATGGATTCTCGAGAAATCCATGCAGAAACATGGGTTTTTG
(2) CCGGCCTTACCACTATCAGAGAGTACTCGAGTACTCTCTGATAGTGGTAAGGT
(3) CCGGCTGTCCAATGTCAACCGGAATCTCGAGATTCCGGTTGACAT TGGACAGTTTTTG
(4) CCGGGCACACAATAACTTGGACCTACTCGAGTAGGTCCAAGTTATTGTGTGCTTTTTGTTTTG
(5) CCGGCATCCGTATGAGCTTCGTCAACTCGAGTTGACGAAGCTCATACGGATGTTTTTG].

shRNAs were used in a Smad3 shRNA cocktail (shRNAs 1-5), designated Smad3 shRNA-1, or individually [shRNA(2) designated Smad3 shRNA-2 and shRNA(3) designated Smad3 shRNA-3]. Skeletal muscle was infected by IM injection of lentiviral particles (˜50,000 TU) with a 28-gauge needle on multiple consecutive days, prior to or coincident with CTX-1 injury. To examine cell proliferation, 50 μl of 10 mM BrdU was injected IP at 3 days post-injury. Tissues were analyzed for regenerative responses and transduction levels at 5 days post-injury via cryo-sectioning of whole tissues, or Western blotting of satellite cell lysates (described above). Transcript levels were analyzed using SuperScript RT-PCR kit (Invitrogen) for amplification of Smad3 [F=CTGGCTACCTGAGTGAAGATGGAGA, R=AAAGACCTCCCCTCCGATGTAGTAG] and GAPDH [F=TGAGGCCGGTGCTGAGTATGTCGTG, R=TCCTTGGAGGCCATGTAGGCCAT]. Amplification products (25-40 cycles on BioRad iQ5) were examined and confirmed for predicted molecular weights on EtBr-stained 2% agarose gels.

### Example IV

### Immunocytochemistry and Histological Analysis

Muscle tissue was treated in a 25% sucrose/PBS solution, frozen in OCT compound (Tissue Tek), cryo-sectioned at 10 m (Thermo Shandon Cryotome E) and immunostained as previously described. Immunostaining or Hematoxylin and Eosin staining were performed on cryosections. For indirect immunofluorescence, sections were permeabilized in [PBS, +1% FBS, +0.25% Triton X-100], incubated with primary antibodies for 1 hr at room temperature in PBS, +1% FBS, and then with fluorophore-conjugated, species-specific secondary antibodies for 1 hr at room temperature (1:500 in PBS, +1% FBS). pSmad3 and BrdU-specific immunostaining required additional nuclear permeabilization and DNA-chromatin denaturation with 4N HCL. Nuclei were visualized by Hoechst staining for all immunostains. Biologically-active TGF-β and myostatin proteins (immunodetectible ligands cleaved from the latent complex)^{14,29} were also examined. Samples were analyzed at room temperature with a Zeiss Axio Imager A1, and imaged with an Axiocam MRc camera and AxioVision software.

### Example V

### ChIP Assays and Real-Time qPCR/PCR

Isolated satellite cells were treated with TGF- only, activation of Notch only, TGF-β1/Notch together, Notch inhibition (GSI) or untreated (as described above). After 24 hours of culture, satellite cells were fixed with 1% PFA and ChIP assay was performed according to manufacturer's guidelines (Upstate). Fragments of ˜500 bp were produced by shearing DNA with attached proteins (confirmed by EtBr-stained gels), and precipitated with antibodies to DNA-bound protein. Proteins that co-precipitated with pSmad3 were analyzed by Western blot, using indicated antibodies. DNA that co-precipitated with pSmad3 was analyzed using primers specific for the 5′ gene regulatory regions of p15, p16, p21 and p27 [p15 F=GTTCAGGGCGTTGGGATCT, R=TCACCGAAGCTACTGGGTCT; p16 F=GTCACACGACTGGGCGATT, R=GTTGCCCATCATCATCACCCT and F=GATGACTTCACCCCGTCACT, R=AACACCCCTGAAAACACTGC/GTCCCTCCTTCCTTCCTCTG; p21 F=CCGCGGTGTCAGAGTCTA, R=CATGAGCGCATCGCAATC; p27 F=AGCCTACGCTCCGACTGTT, R=ACTTCTGCGACTGCACACAG and F=CTAGCCACCGAAGCTCCTAA, R=AGTTCTGCGACTGCACACAG and F=CTGGCTCTGCTCCATTTGAC, R=GGCTCCCGTTAGACACTCTC]. GAPDH primers (see above) were used as control for Smad3 non-enriched genomic regions. Primers were designed with OligoPerfect Designer (Invitrogen). For real-time qPCR, samples were analyzed using a Bio-Rad iQ5 real-time PCR detection system, with iQ5 optical system software. For PCR, samples were amplified with Platinum Taq DNA Polymerase (Invitrogen) and analyzed on a BioRad iQ5 system. Following 40-55 cycles of amplification, fragments produced from each primer set were examined and confirmed for their predicted molecular weights on EtBr-stained 2% agarose gels. 55 cycles of amplification were used for negative control PCR reactions.

### Example VI

### Methods and Compositions for Studies of Human Cells

For studies of human cells described herein, young (22.6 years, range 21-24 years) and old (71.3 years, range 68-74 years) volunteer male subjects were screened by a physician to exclude persons with cardiovascular disease, diabetes, neural-or musculoskeletal disease, inflammatory or pulmonary disorders and any known predisposition to deep venous thrombosis. Only healthy, non-medicated individuals were included in the study. Subjects were moderately active and none of the subjects had previously participated in systematic strength training. Local Ethics Committee approved the conditions of the study.

Maximal muscle contraction strength was measured (1 KHz) for the quadriceps femoris muscle in vivo, as the peak knee extension torque exerted during maximal voluntary concentric muscle contraction in an isokinetic dynamometer (KinCom). Total contractile work in the range of motion (90° to 10°, 0°=full knee extension) was determined as the time-integral of contractile power production, where power was calculated by the instantaneous product of muscle torque and joint angular speed, the latter expressed in radians.

Bilateral muscle samples from 21 individuals (10 young, 11 old) were obtained from the middle portion of m. vastus lateralis, utilizing the percutaneous needle biopsy technique of Bergstrom (Bergstrom J et al (1976) Clin Sci Mol Med Suppl 51:589-599.). After dissecting the muscle samples of all visible blood, adipose and connective tissue, the muscle samples were oriented in embedding medium (Tissue Tec), frozen in isopentane cooled with liquid nitrogen and stored at −80° C. Subsequently serial transverse sections (10 μm) were cut in a cryotome at −20° C. and stained for myofibrillar ATPase at pH 9.4 after both alkaline (pH 10.3) and acid (pH 4.3 and 4.6) pre-incubations. All samples of each individual person were stained in the same batch to avoid inter-assay variation. For the determination of muscle fiber size, only truly horizontal fibers were used, with a minimum of 100 fibers included for the analysis. A video-scope consisting of a microscope (Olympus BX 50) and color video camera (Sanyo high resolution CCD) in combination with Tema Image-analyses System (Scanbeam Denmark) were used to calculate the mean fiber area of the muscle fibers.

Myofiber and satellite cell cultures were isolated from basal state human biopsies, in a manner similar to those previously described for the mouse model (Conboy et al. (2003) Science 302:1575-1577). Whole muscle biopsies were prepared for myofiber fragments by enzymatic digestion −37° C. in DMEM (Invitrogen, Carlsbad, Calif.)/Pen-Strep (Invitrogen)/0.2% Collagenase Type IIA (Sigma), trituration, and multiple sedimentation and washing procedures. Isolated satellite cells and myofibers were resuspended in growth medium (Ham's F10 nutrient mixture—Mediatech, Inc., Herndon, Va.), 10% BGS, 5 ng/ml hbFGF (Invitrogen) and 1% Pen-Strep, and cultured on Matrigel (diluted 1:250). Blood was collected from subjects for sera isolation. Briefly, blood cells were coagulated followed by centrifugation at 8,000 rpm, 4° C. in a microfuge for 3 minutes.

For isochronic and heterochronic systemic cultures, myofiber explants or isolated satellite cells were cultured in the presence of 10% young or old sera. Isochronic cultures were performed by culturing isolated satellite cells with their respective donor serum. Heterochronic cultures used pooled sera, derived from specific donor cell lines examined. Following specific time-point incubation, cells were fixed in 70% EtOH/PBS for immunodetection.

Isolated muscle tissue was frozen in OCT compound (Tissue Tek) and cyrosectioned as 10 μm slices (Thermo Shandon Cryotome E). Immunostaining and haematoxylin and eosin staining were performed using methods known in the art and as described in Conboy et al. ((2003) Science 302:1575-1577) and Carlson et al., ((2008), Nature, 454:528-532). Or indirect immunofluorescence assays, sections were permeabilized in PBS, +1% FBS, +0.25% Triton X-100 and incubated with primary antibodies overnight at 4° C., PBS, +1% FBS. Secondary staining with fluorophore-conjugated, species-specific antibodies was performed for 1 hour at room temperature (1:500 in PBS, +1% FBS). Nuclei were visualized by Hoechst staining, and samples were analyzed at room temperature with a Zeiss Axio Imager A1 and imaged with an Axiocam MRc camera/AxioVision software.

For Western Blot analysis, whole skeletal muscle lysates were prepared in lysis buffer (50 mM Tris, 150 mM NaCl, 1% NP40, 0.25% sodium deoxycholate and 1 mM EDTA, pH 7.4) with addition of protease inhibitor cocktail (Sigma) and 1 mM PMSF. Phosphatase activity was inhibited by 1 mM sodium fluoride and 1 mM sodium orthovanadate. For most assays, 30 μg protein extracts were run on pre-cast SDS PAGE gels (BioRad). Primary antibodies were diluted in 5% non-fat milk/1× PBST, and nitrocellulose membranes were incubated with antibody mixtures overnight at 4° C. HRP-conjugated secondary antibodies (Santa Cruz Biotech) were diluted 1:1000 in 1× PBST/1% BSA and incubated for 1 hour at room temperature. Blots were developed using Western Lightning ECL reagent (Perkin Elmer) and analyzed with Bio-Rad Gel Doc/Chemi Doc Imaging System and Quantity One software. Results were quantified by digitizing the data and normalizing pixel density of examined protein by actin-specific pixel density.

Quantified data was expressed as mean±s.d. Significance testing was performed using one-way analysis of variance, with an alpha level of 0.01-0.05 to compare data from different experimental groups. A minimum of three replicates were performed for each described experimental condition. In vitro experiments typically analyzed 5 young and old individuals per assay unless otherwise noted.

### Example VII

### Comparison of Muscle Regeneration and Functional Recovery

Cells were obtained and analyzed according to the methods described in Example VI above.

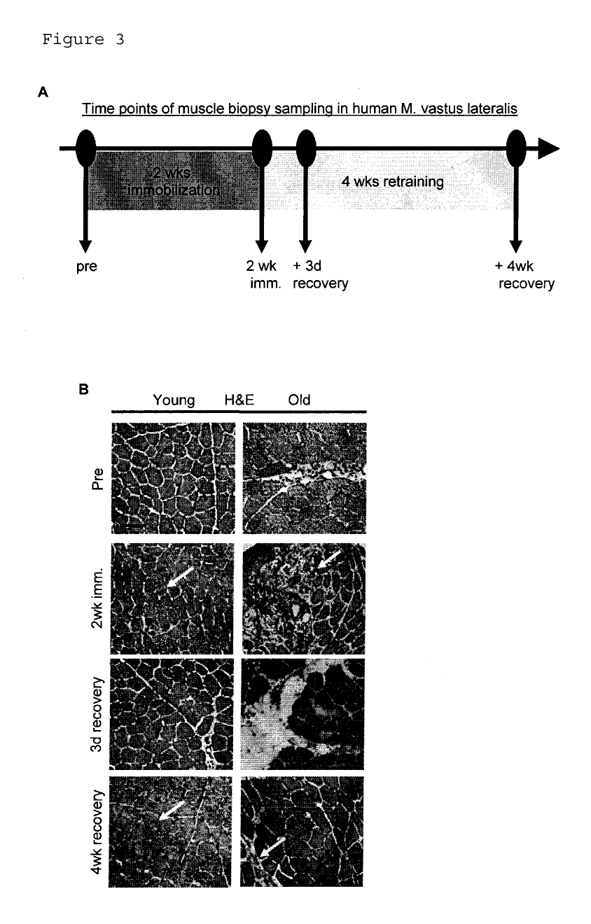

In order to compare muscle regeneration success and functional recovery between young (20 year old) and aged (˜70 year old) human subjects, myofiber atrophy was induced by immobility (cast application for two weeks), followed by acute exercise (loading) of skeletal muscle after cast removal (for 3 days and for 4 weeks), aimed to promote muscle regeneration and functional improvement in strength and agility. Muscle biopsies were collected prior to immobility (basal level), after 2 weeks of immobility (induced atrophy), 3 days after cast removal (initiation of regeneration and functional recovery). The scheme of this experimental setup is depicted in FIG. 3A.

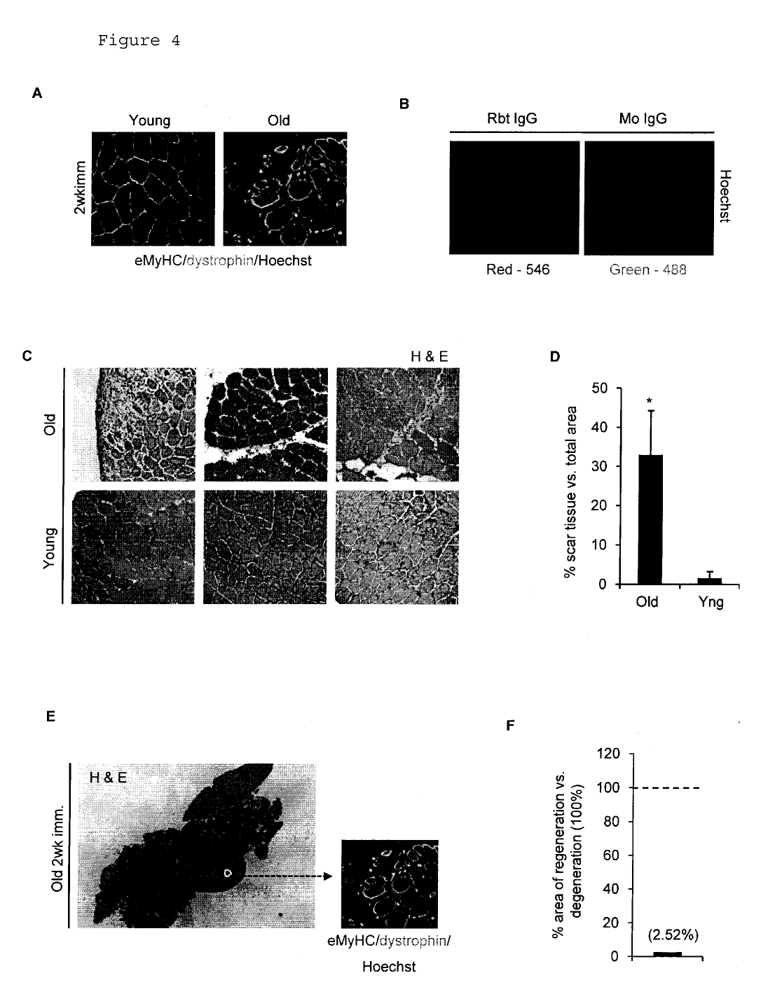

To determine whether muscle maintenance was age-dependent under the conditions of mobility, immobility-atrophy and loading-recover, 10 μm cryosections derived from the young and old muscle biopsies at the indicated time points were analyzed. As shown in FIG. 3B, the muscle histology was markedly different between young and aged individuals, in the basal (‘Pre’) state (prior to immobility) and was particularly different during the immobility and recovery periods of immobility-induced atrophy. As compared to young, the old human muscle fibers were uneven in size and less numerous before immobility (Pre). Old myofibers underwent severe degeneration during immobility, as compared to mild degeneration of young myofibers (2 week). Additionally, old myofibers, but not young, exhibited a persistent inflammatory response and scar formation at both 4 days and 4 weeks of recovery (FIG. 3B). The immobility-caused myofiber degeneration in old individuals was highly pronounced and similar to pathological degenerating muscle, with its typical clusters of new eMyHC+myofibers and broken sarcolemma evidenced by uneven pattern of dystrophin, e.g., in cases of congenital myopathies. In contrast, the acutely deteriorating muscle clusters were absent and intact dystrophin+sarcolemma were typical in young muscle, suggesting better tissue maintenance during immobility (FIG. 4). These data suggest that both maintenance of immobilized skeletal muscle and regeneration of atrophic myofibers after cast removal become inefficient in older individuals, manifested as the replacement of functional tissue by fibrotic scar tissue.

Based on the quantification of quadriceps muscle cross sectional area (MRI), old muscle fibers were much smaller than young in the ‘pre’ state and, as compared to young, the size of old muscle fibers was not efficiently recovered following cast removal and exercise. Histology and muscle size data were further confirmed and extrapolated by functional studies on muscle concentric/isometric strength and total muscle contraction work, thus establishing that while both young and aged individuals recovered close to basal levels of these functional parameters by exercising after immobility, old muscle always remained weaker than young.

Without being limited by theory, one possible mechanism for age-specific decline in muscle fiber maintenance, repair, size and strength in different conditions, including normal muscle use and during recovery from immobility-induced atrophy, may be a result from age-specific alterations in many different parameters, including without limitation innervation and vascularization as well as the lack of muscle fiber regeneration.

To test whether decline in maintenance and repair of muscle functional unit (myofiber maintenance via resident satellite cells) is a factor causing lack of old human muscle regeneration, strength and agility, loss of muscle stem cells and/or age-specific decline in satellite cell activation was investigated.

Different experimental systems and standards for what is considered to be a satellite cell are used in the field, and many tests used in the field involve cardiotoxin or dry ice, which are not physiological agents of human muscle repair and remodeling. In order to clarify the situation, a model of human myofiber regeneration after atrophy was used to compare the numbers of quiescent muscle satellite cells associated with undamaged myofibers with the numbers of satellite cells activated by myofiber deterioration. Satellite cells were quantified using immuno-detection of three different markers: Pax7, NCAM and M-Cadherin, in young and old human skeletal muscle cryosections.

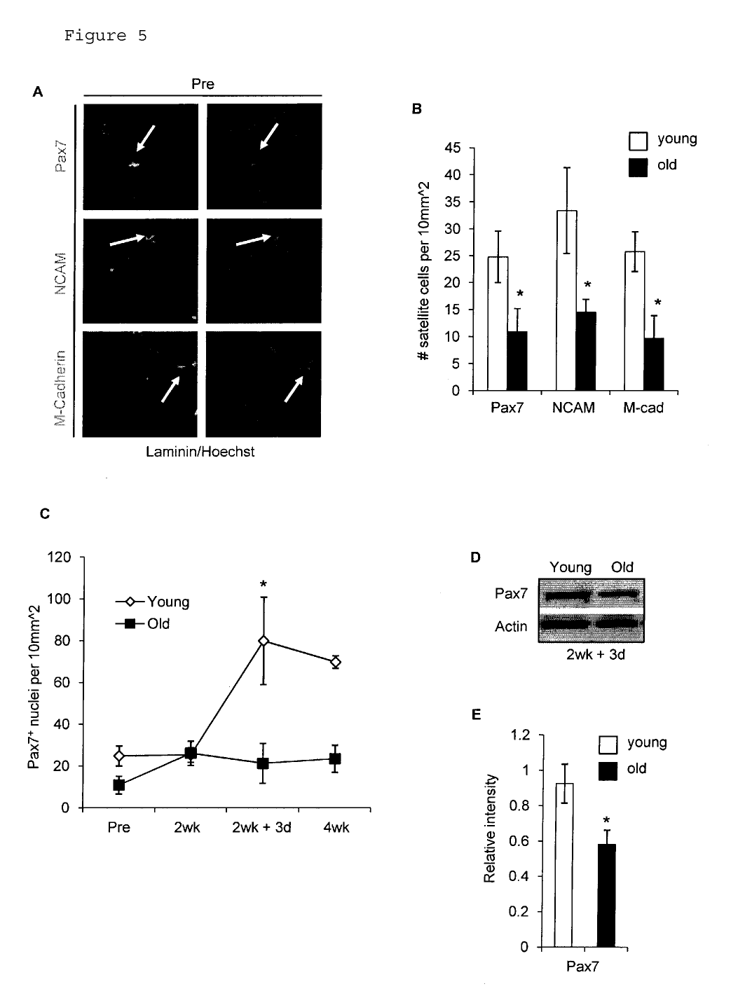

As shown in FIG. 5A-C, aging produces an approximately 2-fold decline in the number of satellite cells endogenous to old muscle in the basal state. The number of the old myogenic cells increases slightly during old muscle immobility (2 weeks), which is consistent with the ongoing degeneration and attempts at regeneration of old tissue, as shown in FIG. 3B and FIG. 4. There was a pronounced, approximately 4-fold age-specific decline in the expansion of satellite cells in response to exercise after the immobility-induce atrophy (3 days and 4 weeks), FIG. 5A-C. The age-specific decline in numbers of Pax7 myogenic cells during exercise after immobility was also confirmed by Western Blotting (FIG. 5D, E) performed on whole muscle protein isolates using actin as a loading control. Data in FIG. 5E are means±s.d. n=10 for immunostained cryosections, n−6 for Western Blot analysis. These data suggest that stem cell activation significantly declines with age in humans, which may contribute to the lack of muscle maintenance and repair and to the replacement of myofibers by fibrous scar tissue in older humans (FIG. 3 and FIG. 5).

### Example VIII

### Investigation of Notch Signaling in Young and Old Human Tissue

Cells were obtained and analyzed according to the methods described in Example VI above.

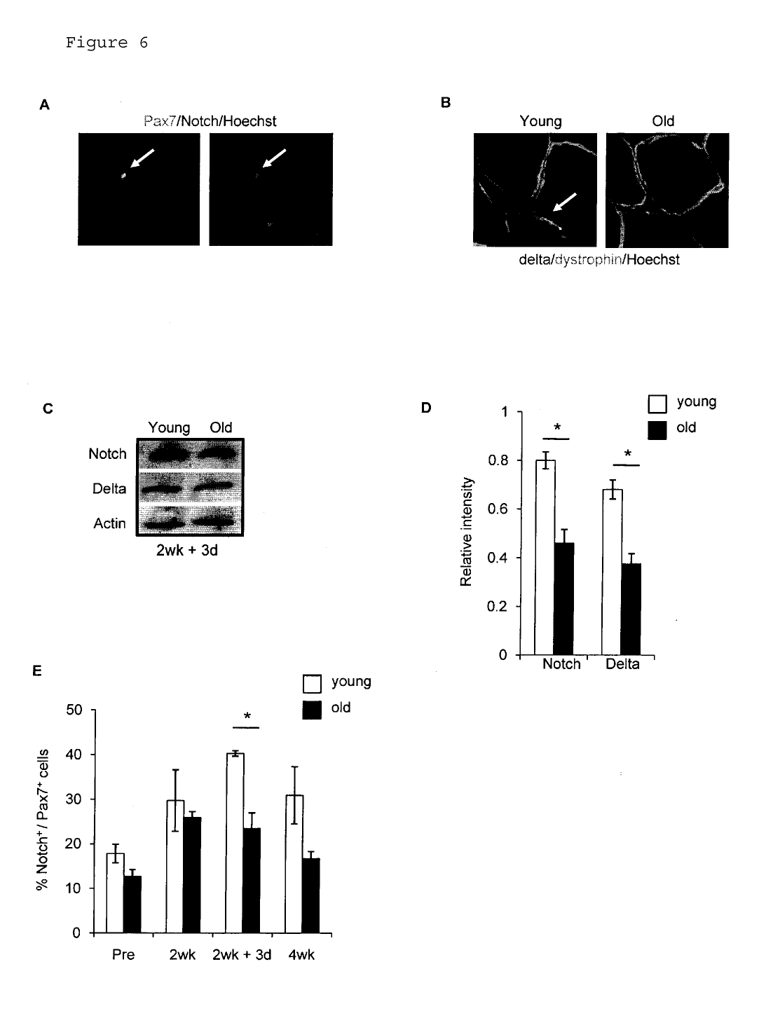

To determine whether Notch activation is lacking in old human satellite cells associated with aged muscle in vivo, Notch and Pax7 co-immunodetection experiments in cryosections of human muscle biopsies were performed. As shown in FIG. 6A, nuclear active Notch can be detected in Pax7+myofiber-associated cells. In addition, levels of the Notch ligand Delta are diminished in old myofibers as compared to young myofibers (FIG. 6B). Decline in active Notch and its ligand Delta is observed in Western blot analysis of young and old human muscle (FIGS. 6C and D). Data in FIG. 6 are n=10-15 for immunodetection of cryosections and n=6 for Western blot analysis.

Expansion of myogenic cells in regenerating human muscle (during exercise after immobility) is positively correlated with levels of active Notch. Numbers of Pax7+/Notch active cells were low in the basal state and during immobility, but greatly increased at three days post-immobility in young but not in old human muscle (FIG. 6E). Numbers of Pax7+/Notch active human satellite cells declined after several weeks of regeneration, when the differentiation process follows initial cell expansion (FIG. 6E). Accordingly, while there were still more Pax7+/Notch active cells in the young as compared to old human muscle at four weeks post-immobility, the total numbers of activated myogenic cells decline in both young and old tissue (FIG. 6E). These results suggest that Notch regulation becomes altered during human aging in skeletal muscle and suggests the importance of Notch for expansion of human satellite cells.

### Example IX

### Investigation of TGF-β/pSmad Pathway Signaling in Young and Old Human Tissue

Cells were obtained and analyzed according to the methods described in Example VI above.

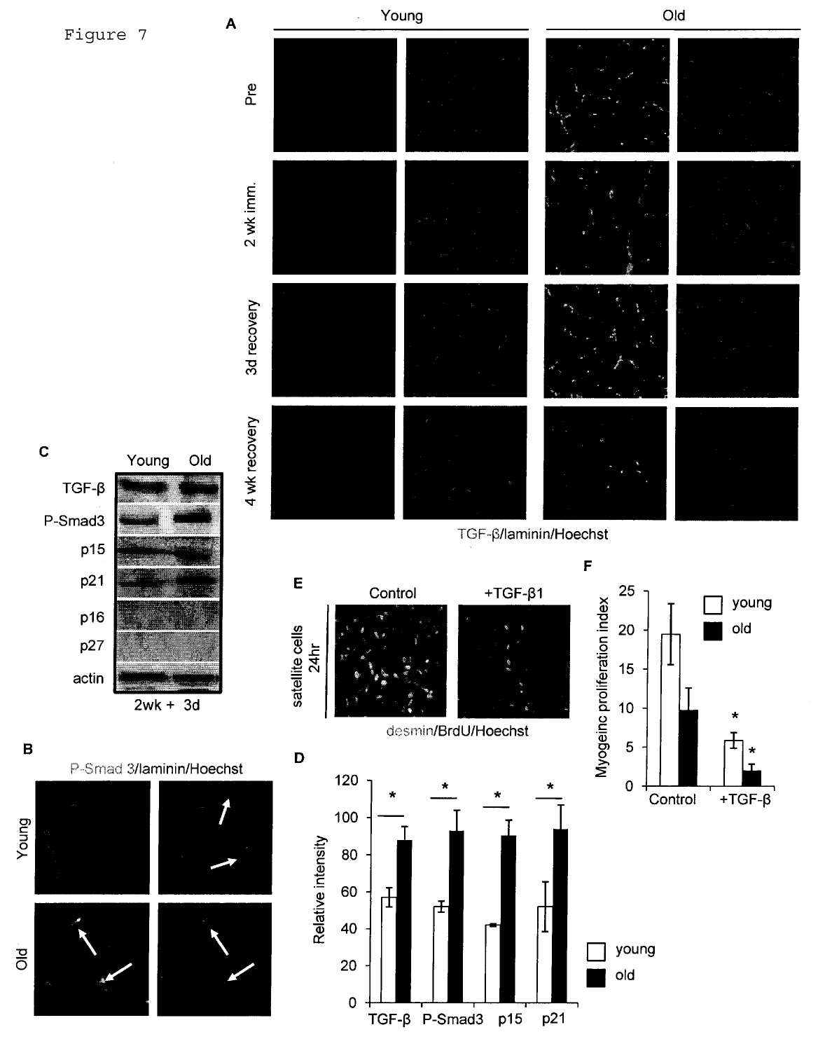

Molecular signatures of aging within the muscle stem cell compartment are conserved between mouse and human (FIG. 7). As compared to young, old human muscle fibers contain higher levels of TGF-β, which associates with the laminin-rich basement membrane of the satellite cell microniche (FIG. 7A). Accordingly, levels of nuclear pSmad3 (the transcriptional factor that is activated by TGF-β signaling) are excessive in old human satellite cells, as compared to young (FIG. 7B). Western blot analysis of young and old human muscle shows that the levels of TGF-β, pSmad3 and CDK inhibitors, p15 and p21 (known to be induced by TGF-β signaling and reduced by active Notch) are all higher in the old, as compared to young human muscle.

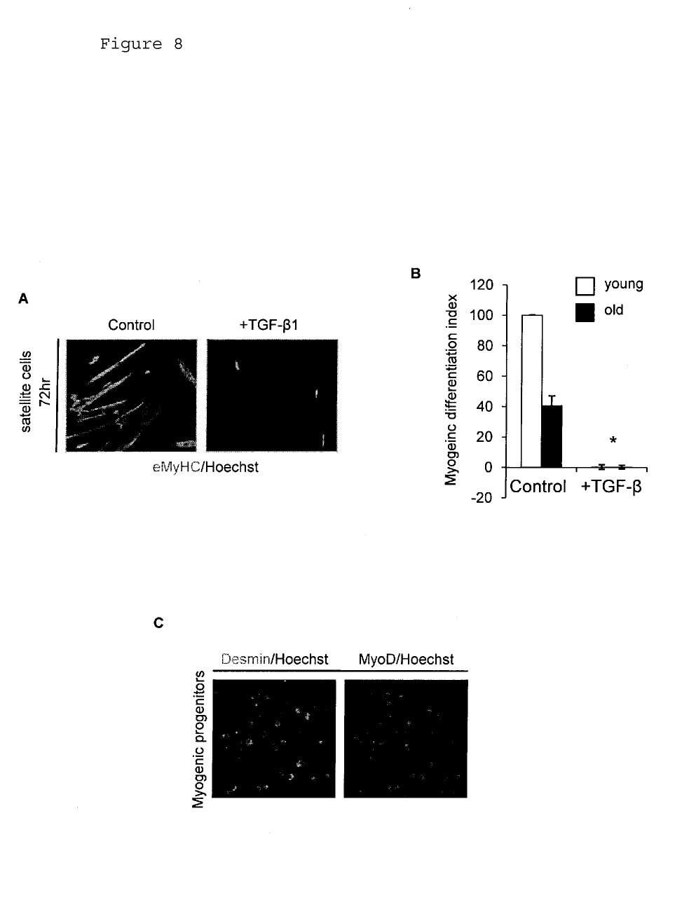

To examine effects of TGF-β on myogenic properties of human muscle stem cells, exogenous molecule was added to young and old human satellite cells in culture. Myogenic capacity was determined based on numbers of fusion-competent, proliferating myoblasts, e.g., cells that rapidly (in approximately 2 hours) incorporate BrdU, co-express desmin and MyoD and fuse into eMyHC+myotubes when transferred to mitogen-low medium. As shown in FIGS. 8E and F, the myogenic regenerative potential was dramatically reduced by TGF-β1, thus confirming its role as a negative regulator of muscle regeneration. Satellite cells isolated from young humans exhibited higher myogenic potential as compared to satellite cells derived from old people in these 24 hour isochronic cultures where the age of cells is matched with the age of sera (FIGS. 7E and F). Higher magnitudes of age-specific deficiency in myogenic responses were observed in 7-day isochronic human satellite cell cultures (FIG. 8). Additionally, similar to findings in the mouse model, young human satellite cells had diminished regenerative responses when cultured in the presence of old sera in heterochronic co-culture assays (FIG. 8). Conversely, the myogenicity of old satellite cells was improved when cultured in young sera.

### Example X

### Investigation of MAPK Pathway Signaling in Human Satellite Cells

Cells were obtained and analyzed according to the methods described in Example VI above.

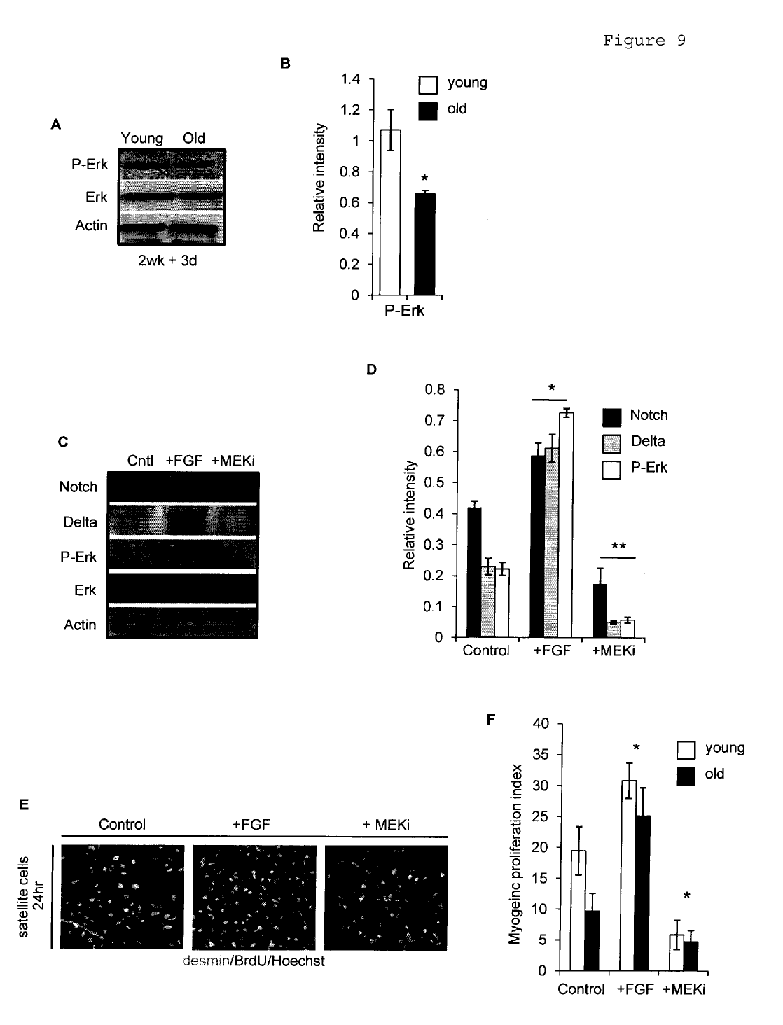

Western Blot analysis of young and old human muscle demonstrated that MAPK signaling strength is down-regulated with age (FIGS. 9A and B). The levels of Delta, amounts of active Notch and the efficiency of myogenic responses in human satellite cells cultured in the presence of agonists and antagonists of MAPK pathway were investigated. MAPK agonist FGF-2 induced Delta and active Notch, while specific inhibitor of MAPK (MEK inhibitor) significantly attenuated Delta and active Notch levels (FIG. 9C, D). Levels of pERK (a downstream effector of MAPK) were induced by FGF-2 and reduced by MEK inhibitor, thus validating the success of experimental modulation of MAPK (FIG. 9C, D). Myogenic regenerative capacity of young and old satellite cells was significantly enhanced through forced activation of MAPK, and even young satellite cells failed to produce proliferating fusion-competent myoblasts when MAPK was experimentally inhibited (FIG. 9E, F). Consistent with the data for control isochronic cultures, young satellite cells outperformed old satellite cells (FIG. 9E, F).

To confirm that MAPK effect on human satellite cell responses was through Notch activation, MAPK was activated in the presence of a Notch antagonist, gamma secretase inhibitor (GSI). Inhibition of Notch by GSI precluded satellite cell regenerative responses even when MAPK was induced by FGF-2, suggesting that positive regulation of satellite cell myogenicity by MAPK acts up-stream of Notch activation. These data suggest that MAPK pathway is an age-responsive positive regulatory of Notch in human muscle.

### Example XI

### Investigation of Forced Notch Activation in Human Satellite Cells

Cells were obtained and analyzed according to the methods described in Example VI above.

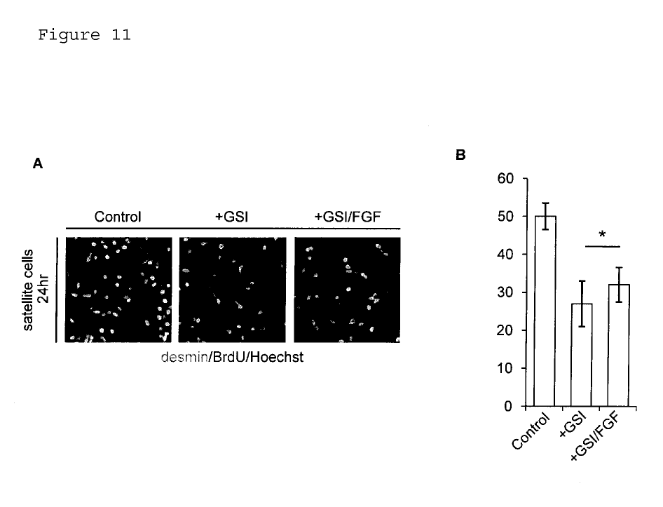

Experimental activation of Notch receptor by exogenous ligand Delta and forced inhibition of Notch by GSI were performed for seven days of culture (control activation and inhibition of Notch shown in FIG. 11; additional experiments on control young and old isochronic cultures shown in FIG. 8). Myogenic responses were measured based on the generation of proliferating fusion-competent myoblasts. These data suggest that Notch is a molecular determinant of human myogenic responses and may be required for productive myoblast generation by young stem cells and capable of rescuing myogenesis of aged human satellite cells even in old systemic milieu.

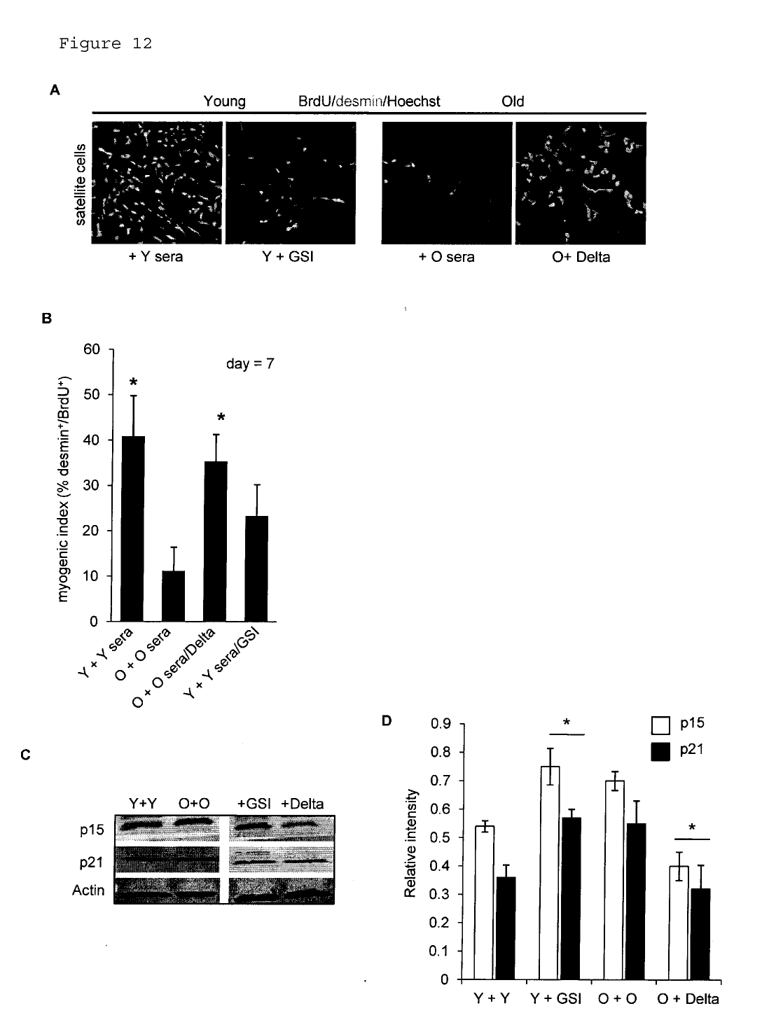

Levels of CDK inhibitors p15 and p21 were compared in satellite cells cultured in control isochronic conditions and in cells with forced activation and inhibition of Notch. Results of Western Blotting shown in FIG. 12C and D suggest that levels of p15 and p21 are higher in old satellite cells as compared to young, in agreement with age-specific elevation of these CDK inhibitors in human muscle in vivo, FIG. 7C. Thus manipulating levels of such molecules may be another way to tune genetic circuitry to establish levels of signaling that correspond to young versus old cells.

### Example XII

### Investigation of Myogenic Responses from Initially-Equivalent Satellite Cell Populations

An initially equivalent population of young and old satellite cells (625 cells seeded per well for Yng+Ys, Old+Ys, Old+Os and Ynd+Os) yielded different numbers of myogenic progeny or myoblasts (significantly higher in the young versus old after seven days of culture). This age-specific decline is reversed if the aged satellite cells are cultured with young human serum (Old+Ys). (Ys=young serum and Os=old serum) Thus, even with equivalent and clearly myogenic satellite cellular pools, the regenerative outcome of the old cells in an old environment is worse than young cells in a young environment (see Table I below).

TABLE I

| In vitro condition | | % desmin+/BrdU+ |
|---|---|---|
| Yng + | Ys | 72.4 |
| Old + | Ys | 67.9 |
| Old + | Os | 35.0 |
| Yng + | Os | 23.5 |

### Example XIII

### Comparison of TGF-β1 and Wnt Levels in Aged Serum

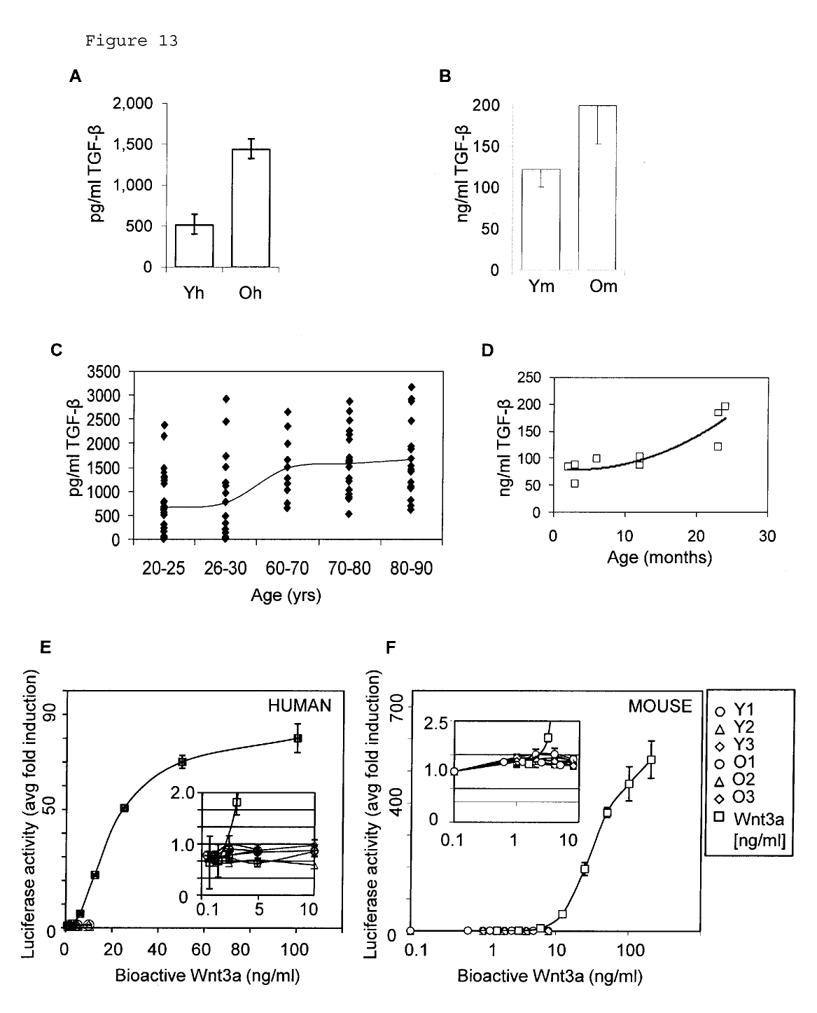

Levels of TGF-β1 and Wnt in the serum of young and old humans and mice, using ELISA and biological activity assays were measured. A significant (p<0.0006) rise in the systemic TGF-β1 levels was observed in old human serum, where the median serum level from young individuals (n=51, 20-35 years) was only two thirds that of old individuals (n=51, 65-90 years) (FIG. 13A). Additionally, two fold elevation of systemic TGF-β1 levels were found in old mice as compared to the young counterparts (FIG. 13B), which was also corroborated by the TGF-β1 biological activity assay, using the plasminogen activator inhibitor-1 (PAI-1) promoter luciferase-reporter. TGF-β receptor I levels increase approximately 3 to 4 fold with age, suggesting a compounding effect on signaling of elevated ligand and receptor.

An age-specific incline curve of systemic TGF-β1 was constructed, which revealed a continuum in elevation of this cytokine with age, both in mice and in humans (FIG. 13C). Interestingly, in humans, TGF-β1 plateaus at its highest systemic levels between the 6th and 9th decades of life; thus, at the onset of regenerative decline TGF-β1 is already high and is maintained as such during the progressive regenerative failure for at least 30 years (FIG. 13D). In mice, TGF-β1 levels increase between 12 month (early post-reproductive age analogous to 5th-6th decades in people) and 24 month (analogous to 8th-9th decades in people), FIG. 13C.

Using an analogous luciferase-reporter assay, systemic levels of biologically active Wnt were measured, since an age-specific increase of circulating biologically active Wnt (from ˜10 ng/ml in young to ˜20 ng/ml in old sera) has been reported. These experiments robustly demonstrated that Wnt was undetectable in either young or old blood sera derived from either humans (FIG. 13E) or mice (FIG. 13F). Specifically, the levels of Wnt in young or old human or mouse sera were significantly lower than 5 ng/ml (based on recombinant Wnt standard curve), and the relative luciferase activity values were actually higher for young mouse sera, as compared to old (FIG. 13E, F). These results show that an age-specific increase of circulatory Wnt does not occur, while the age-specific elevation of systemic TGF-β1 is readily detectable and furthermore, is evolutionary conserved between mice and humans.

### Example XIV

### Role of TGF-β1 and Wnt in Age-Specific Inhibition of Muscle Stem Cell Responses

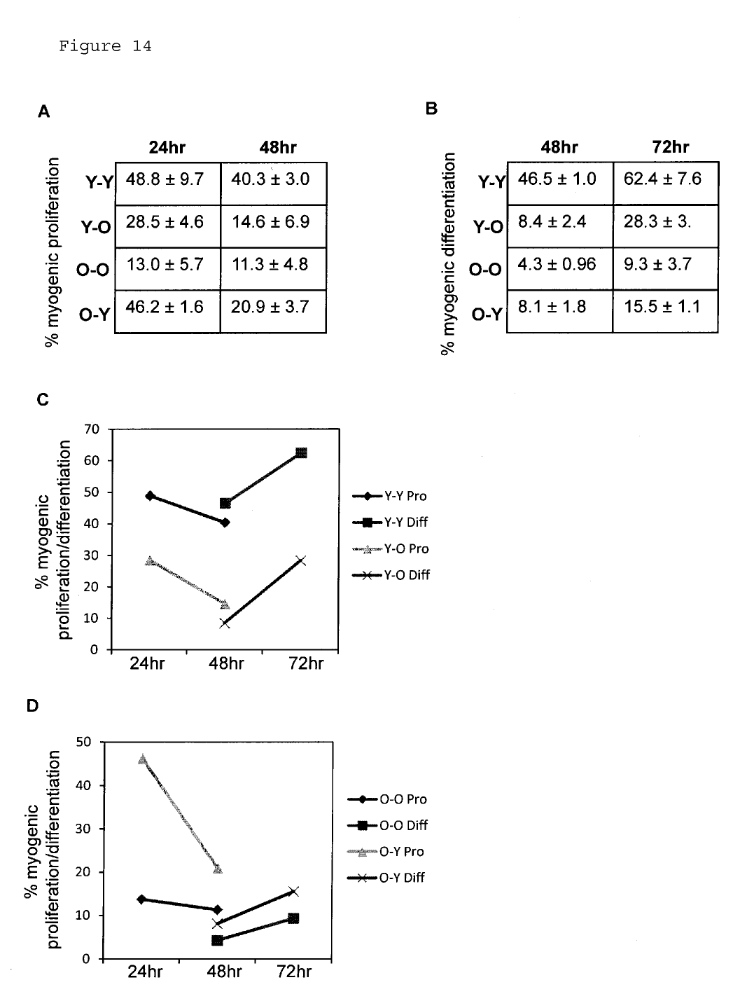

To examine the relative role of circulating TGF-β1 in the age-specific inhibition of muscle stem cell responses, TGF-β1 was depleted from young and old mouse sera and then added back in a range of concentrations. Muscle stem cells derived from young and old mice were cultured in the presence of young and old mouse sera that differed only in the levels of TGF-β1, and the regenerative responses of these cells were assayed. Serum was depleted of TGF-β1 by incubation with a TGF-β1 specific antibody (or an isotype-matched control IgG) followed by binding and removal of the TGF-β1-antibody complexes (or IgG control antibody complexes, e.g. control sera) using protein G-coated agarose beads. The success in the TGF-β1 depletion was confirmed by ELISA. The myogenic regenerative potential of muscle stem cells was quantified based on their ability to rapidly (overnight) generate de-novo myogenic lineages: BrdU-incorporating desmin+myoblasts (myogenic proliferation) that differentiate into post-mitotic eMyHC+myotubes (myogenic differentiation). The time course of such de-novo in vitro myogenesis that recapitulates the in vivo muscle repair is shown in FIG. 14A-D. Myogenic proliferation (at 24-28 hours of culture) and myogenic differentiation (at 48-72 hours of culture) were consistently higher for young satellite cells as compared to old satellite cells and in young sera as compared to old sera at all time points (FIGS. 17 and 18). The non-myogenic, desmin negative proliferating fibroblasts (known to contaminate satellite cell preparations at ˜2-5% and to expand in the presence of old sera or high levels of TGF-β (Carlson et al., 2008) were less numerous in cultures with experimentally calibrated levels of TGF-β1 (FIG. 15) and also declined in cultures with exogenous Wnt 3A (FIG. 16A).

The productive myogenic proliferation of both young and old muscle stem cells was restored in TGF-β1-depleted old serum, but only when low levels of recombinant TGF-β1 were added and similar phenotype was observed with TGF-β1-depleted young serum (FIG. 15A-C). These data demonstrate that the biologically active TGF-β1 contained in the serum (both young and old) directly inhibits satellite cell responses, that TGF-β1 alone is sufficient for such inhibition, and that certain low levels of TGF-β1 are actually required for productive myogenic responses.

With respect to myogenic differentiation, the response of young cells also peaked in TGF-β1-depleted serum to which low levels of recombinant TGF-β1 were added (FIG. 15D). In contrast, myogenic differentiation of old satellite cells was improved by the TGF-β1-depletion from sera, as well as in the low range of recombinant TGF-β1 and their general myogenic differentiation was diminished as compared to young satellite cells (FIG. 15E). Since, myogenic differentiation is assayed at 48-72 hours of culture and aged satellite cells have elevated production of TGF-β1, these data are suggest that TGF-β1 produced by old cells in culture counteracts the beneficial effect of depleting TGF-β1 from mouse sera (FIGS. 17 and 18).

Recombinant TGF-β1 was added at a range of concentrations to these mouse sera (depleted from endogenous TGF-β1) and assayed the regenerative responses of muscle stem cells, as above. As shown in FIG. 15, at 1-5 ng/ml (and higher) concentrations of TGF-β1 alone suffices for the inhibition of myogenic satellite cell responses, without experimental manipulation of other cytokines. Comprehensively, the curves of TGF-β1-imposed decline in the myogenic cell proliferation and differentiation demonstrate that there is an inhibitory threshold of TGF-β1 levels (FIG. 15A-E), where either too low or too high levels of this systemic cytokine inhibit muscle stem cell regenerative responses. The levels of TGF-β1 that are permissive for myogenic responses correspond to the range of bioactive TGF-β1 found in young blood sera, while the inhibitory range corresponds to the levels typical of aged circulation. The threshold mode of TGF-β1-imposed inhibition of muscle repair established here, also correlate well with the results of heterochronic parabiosis.

These data suggest that, without being bound by theory, the pronounced inhibitory activity of the aged systemic milieu that directly interferes with the regenerative potential of muscle stem cells operates through a TGF-β1-dependent mechanism in a threshold fashion, such that certain circulatory levels permit muscle stem cell regenerative responses, while both below and above levels are prohibitive for satellite cell proliferation and differentiation.

A similar rescue of satellite cell responses in the presence of old serum was reported to be achieved by inhibition of Wnt via FRPs, and exogenous Wnt3A was reported to inhibit satellite cell myogenic responses, in favor of fibroblast trans-differentiation (Brack et al., (2007), Science, 317:807-810). Thus, an investigation was conducted to determine whether Wnt was potentially synergistic with the TGF-β1-dependent attenuation of myogenic potential. Myogenic potential of young and old satellite cells was examined in the following conditions: young serum with exogenous Wnt3A (by itself or simultaneously with exogenous TGF β1) or old serum with exogenous FRP3 (by itself or simultaneously with TGF-β neutralization). As shown in FIG. 16A-E, inhibition of Wnt by exogenous FRP did not rescue the myogenic responses in old serum and exogenous Wnt3A did not reduce myogenic potential in young serum. In fact, consistent with the previously reported pro-myogenic activity of Wnt, Wnt3A enhanced myotube formation. Importantly, in contrast to the induction of fibroblastic cell fate, the presence of non-myogenic fibroblastic cells (e.g. desmin-BrdU+) was not increased, but actually reduced in the presence of exogenous Wnt3A, confirming the pro-myogenic activity of Wnt. Moreover, Wnt modulation did not contribute to the TGF-β1-promoted inhibition of the myogenic responses and in contrast, Wnt3A antagonized TGF-β1 and enhanced generation of fusion-competent proliferating myoblasts by satellite cells (FIG. 16A-E). Additionally, young satellite cells cultured with young sera had less active GSK3β and more inactive GSK3β, as compared to old satellite cells cultured with old sera, suggesting that Wnt signaling is stronger in young conditions (FIG. 16F, G).

### Example XV

### Expression of Dominant-Negative TGF-β Receptor Restores Productive Myogenic Responses to Satellite Cells Exposed to Aged Serum

Myofiber explant cultures and primary myogenic progenitor cells were generated from C57/Bl6 mice using methods known in the art (see e.g., Conboy et al., (2003), Science, 302:1575-1577). Myoblasts were maintained in growth media (Ham's F-10, 20% FBS, 5 ng/ml basic-FGF, and penicillin-streptomycin). Opti-MEM was used to culture myofibers explants in 10% young, 10% old, or 10% young and old mouse serum (5% each). Differentiation media consisted of DMEM and 5% horse serum. Basic-FGF was obtained from Sigma (St. Louis, Mo.) and R&D systems (Minneapolis, Minn.), Opti-MEM was obtained from Invitrogen (Carlsbad, Calif.), and all other cell culture reagents were obtained from Cellgro (Herndon, Va.).

To confirm that both the inhibitory affects of old sera and the rescue effected by TGF-β1 depletion were direct and due to the attenuation of TGF-β receptor engagement, a TGF-β dominant-negative receptor II (TGF-β DN RII) was expressed in satellite cells, and these cells were then examined for their regenerative potential in the presence of old serum. Specifically, young and old satellite cells were activated by muscle injury, and myogenic progenitor cells were isolated three days later and retrovirally transduced with TGF-β DN RII or control vector ex vitro. The dominant negative TGF-β receptor is an inactive kinase that effectively inhibits signaling by active TGF-β ligands (Tang et al., (1999), J. Neurooncol 43:127-135). As shown in FIG. 17, while having no significant effect on satellite cells cultured with young sera, the expression of TGF-β DN RII rescued myogenic proliferation and differentiation in the presence of old sera. The attenuation of TGF-β signaling was confirmed by an absence of nuclear phospho-Smad3 in muscle cells treated with the DN II retrovirus, even when cultured in old serum (FIG. 17C). The ectopic expression of truncated TGF-β DN RII was confirmed by Western Blotting. These data confirm that it is indeed increased signaling through the TGF-β pathway, directly caused by the aged systemic milieu, that inhibits muscle stem cell regenerative capacity.

For retrovirus production and transfection, pMSCV-TβRIIDN retroviral vector used. TβRIIDN was excised from the vector using EcoR I/BamH I, blunted with Klenow (NEB) and inserted into the MoMLV retroviral vector pCLPCGFP. pCLPCGFP was first digested with BamH I following which it was also blunted with Klenow and ligated with the TβRIIDN fragment. The resulting pCLPC TβRIIDN vector was sequenced to confirm its sequence and directionality of insertion. A plasmid made by ligating the blunt ends of the pCLPC vector was used as a control. To produce retrovirus, HEK 293T cells (cultured in Iscove's modified Dulbecco's medium (Hyclone) with 10% fetal bovine serum (Invitrogen) and 1% penicillin-streptomycin (Gibco) and maintained at 37° C. and 5% CO2) were co-transfected with the retroviral vector (pCLPC TβRIIDN or control plasmid) and the helper plasmids pCMV-VSVG, a vector encoding the viral envelope protein from the vesicular stomatitis virus and pCMV-gagpol, a vector encoding the enzymatic and structural retroviral proteins, using a calcium phosphate transfection method. Medium was changed 12 hours after transfection. 48 hours after transfection, the cell culture supernatant containing the virus was collected and purified by ultra centrifugation through 20% sucrose in PBS and resuspended in PBS. The virus also encoded a gene that rendered it resistant against puromycin and it was tittered on 293T cells using WST1 reagent (Roche) following the manufacturer's protocol.

Viral transduction was performed on bulk myofiber explants with activated satellite cells, isolated 3 days post-injury from CTX-injured young and old animals. Cells were infected with TβRIIDN (2×10⁶ TU/ml) or control viruses (1×10⁷ TU/ml) at assay-dependent MOIs, and cultured for 48 hrs in the presence of OPTI-MEM, +5% young or old mouse serum, prior to fixation and analysis. No tetracycline was added.

### Example XVI

### Systemic Delivery of TGF-β Receptor Inhibitor Restores Repair of Old Muscle in Vivo

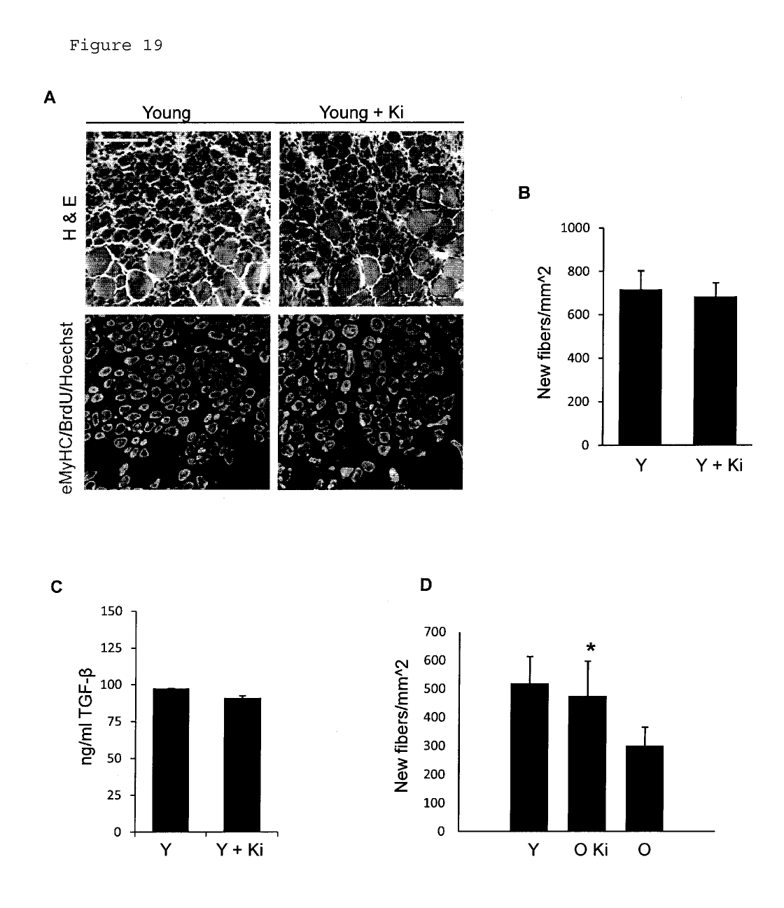

Experiments were conducted to rejuvenate muscle repair in old tissue by attenuating TGF-β pathway activity through systemic pharmacological intervention, in vivo. Three independent attenuators were used: (1) a small molecule inhibitor of TGF-β RI Kinase, (2) TGF-β neutralizing antibody and (3) a decoy composed of the extracellular portion of TGF-β receptor II. These molecules were administered systemically to mice, twice daily. After 10 days of dosage, muscle was injured by CTX, and 5 days after injury and BrdU administration the muscle was harvested, frozen, and examined histologically. As previously published, the efficiency of adult myogenesis and age-specific changes in this process are reliably determined by the detection and quantification of newly-formed myofibers, which are small, express eMyHC and have centrally located, BrdU⁺ nuclei, indicating that these cells are recently generated by previously proliferating fusion-competent myoblasts (Carlson and Conboy (2007), Aging Cell.; Carlson et al., (2008), Nature 454:528-532; Wagers and Conboy, (2005), Cell, 122:659-667). Newly-formed muscle fibers are typically numerous and visible in young regenerating muscle, but less numerous in old muscle, which instead shows more mononucleated cells and scar formation (FIG. 18A). Examination of muscle from mice treated by the systemic administration of the TGF-β RI Kinase inhibitor suggested that regeneration was significantly improved and similar to the regeneration seen in young control animals (FIG. 18A, B) or young animals systemically administered with TGF-β RI Kinase inhibitor (FIG. 19).

Surprisingly, systemic administration of either TGF-β neutralizing antibody or soluble TGF-β receptor II did not result in the rejuvenation of myogenic regenerative capacity in old mice (FIG. 18A, B). To control for the potential differences in the half-life of anti-TGF-β neutralizing antibody as compared to TGF-β RI Kinase inhibitor, constitutive systemic delivery of these molecules was provided by osmotic subcutaneous pumps for four weeks. Interestingly, under these conditions, the anti-TGF-β neutralizing antibody still failed to significantly enhance repair of old muscle, while TGF-β RI Kinase inhibitor promoted the rejuvenated muscle repair (FIG. 18B).

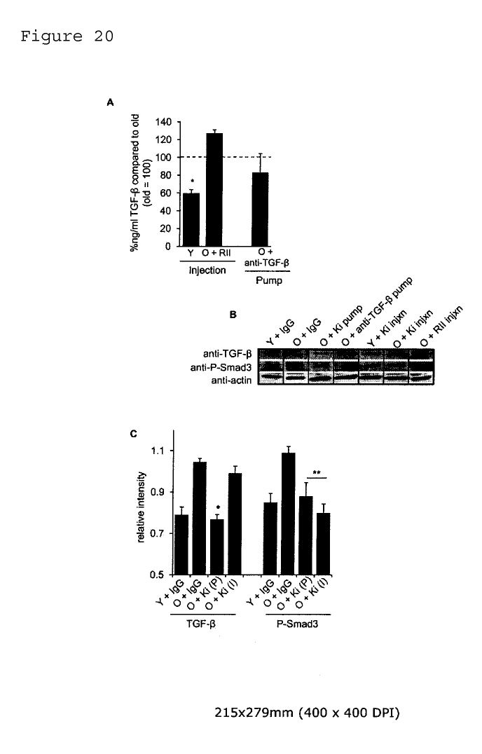

While at first these data seemed contradictory, it was well explained by the ELISA assay on the levels of systemic TGF-β1, which not only corroborated, but in fact, strengthened the above conclusions (FIG. 20A). These data demonstrated that TGF-β1 neutralizing antibody or soluble TGF-β receptor II failed to reduce the circulating levels of TGF-β1 in old mice (FIG. 20A) and accordingly, failed to enhance old muscle regeneration (FIG. 18A, B). Control injections/pumps of goat IgG yielded data typical of young and old untreated mice for muscle repair and TGF-β1 ELISA. Injections of TGF-β RI Kinase inhibitor did not significantly change efficiency of muscle repair in young mice, consistently with already low TGF-β/pSmad signaling (FIG. 19).

To explain the down-modulation of TGF-β1 circulating levels in old mice administered with TGF-β RI Kinase inhibitor through osmotic pumps, it was postulated that the organism-wide prolonged and sustained block of TGF-β/pSmad signaling resulted in a diminished production of TGF-β1 locally in tissues. As shown in FIG. 20B, C, this hypothesis is directly substantiated by the experimental data. Namely, the high levels of pSmad3 and TGF-β1 known to be typical of old muscle are significantly reduced in old mice receiving TGF-β RI Kinase inhibitor through osmotic pumps, but not in old mice administered with anti-TGF-β antibody or soluble receptor (FIG. 20B, C).

Experimental attenuation of Wnt by FRP did not improve the regeneration of old muscle in vivo and did not synergize with the experimental attenuation of TGF-β, which alone sufficed for the rescue of old muscle repair.

These data provide a molecular explanation for the effects of all tested TGF-β attenuators on muscle regeneration in old mice and demonstrate that systemic introduction of a small molecule that inhibits the TGF-β RI Kinase recapitulates the rejuvenating effect of heterochronic parabiosis. These data establish that systemic delivery of a TGF-β RI Kinase inhibitor rejuvenates old muscle repair in vivo and notably, that such enhancement of myogenic potential may require a reduction of TGF-β1 levels and/or signaling strength.

Although described in some detail for purposes of illustration, it will be readily appreciated that a number of variations known or appreciated by those of skill in the art may be practiced within the scope of present invention. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes.

1. A method for enhancing regenerative potential of a cell, said method comprising increasing Notch signaling and attenuating TGF-β/pSmad signaling in said cell.

2. (canceled)

3. The method of claim 1, wherein said increasing Notch signaling and attenuating TGF-β/pSmad signaling comprises introducing a tunable viral construct into said cell.

4. The method of claim 3, wherein said tunable viral construct comprises:
a. a member selected from TGF-β DN RII or Notch ligand, Delta;
b. a tet-inducible promoter; and
c. a tet-responsive trans-activator protein under control of CMV promoter elements, pSmad3 promoter elements, Myf-5 promoter elements, or some combination thereof.

5. The method of claim 1, wherein said cell is a stem cell.

6. The method of claim 5, wherein said stem cell is a muscle stem cell.

7. The method of claim 3, wherein said promoter is stem cell-specific.

8. The method of claim 7, wherein said promoter is an Myf-5 promoter.

9. The method of claim 4, wherein said promoter is TGF-β responsive.

10. The method of claim 9, wherein said promoter is a pSmad3 promoter.

11. (canceled)

12. The method of claim 1, wherein said increasing and said attenuating are in homeostasis, such that said attenuating TGF-β/pSmad signaling results in a concomitant increasing of said Notch signaling.

13. The method of claim 1, wherein said attenuation of TGF-β/pSmad signaling and a concomitant increasing of said Notch signaling self-calibrate, such that modulation of these pathways auto-terminates when healthy normal young levels of TGF-β/pSmad and Notch signaling are reached.

14. The method of claim 3, said method further comprising applying doxycycline to said cell to alter levels of Notch and TGF-β/pSmad signaling.

15. (canceled)
